(19) United States
(12) Patent Application Publication
(10) Pub. No.: US 2020/0046527 A1
(43) Pub. Date: Feb. 13, 2020

(54) WOVEN STENT

(71) Applicants: András Kónya, Houston, TX (US); Jeffery J. Sheldon, League City, TX (US)

(72) Inventors: András Kónya, Houston, TX (US); Jeffery J. Sheldon, League City, TX (US)

(21) Appl. No.: 16/530,647

(22) Filed: Aug. 2, 2019

Related U.S. Application Data
(60) Provisional application No. 62/716,035, filed on Aug. 8, 2018.

(51) Int. Cl. A61F 2/90; A61F 2/07; A61F 2/958

(52) U.S. Cl. CPC A61F 2/90; A61F 2/89; A61F 2/958; A61F 2/07

(57) ABSTRACT

This disclosure provides a self-expandable, woven intravascular device for use as a stent (both straight and tapered) in a variety of anatomical structures, including vessels in the arterial and venous system. The stent provides enhanced structural modifications to conventional woven stents by varying the wire(s) utilized in the woven stent. The stent may be formed from shape memory metals such as nitinol. The stent may utilize multiple, parallel wires instead of a single wire, an increased number of wire pairs and/or pins to vary the mesh size, and/or a bundle of wires (e.g., a plurality of individual wires coupled together) instead of a single wire. The wire mesh may comprise two rows of wire or three rows of wire. The individual wires may each be twisted to provide an overall twist to the wire bundle. The bundle of wires may comprise different sized wires and wires of different materials.

### PRIORITY

This application claims priority to U.S. provisional patent application no. 62/716,035, filed on Aug. 8, 2018, the entire contents of which is incorporated herein by reference.

### BACKGROUND OF THE INVENTION

### Field of the Invention

The present disclosure relates generally to the field of intravascular devices. More particularly, it concerns a self-expanding woven stent, and the methods of making the same, and the apparatus and methods for delivery of the same into a living creature.

### Description of the Related Art

Stents are necessary for both the arterial system and the venous system and are utilized in a wide variety of applications. In general, a stent may generally be characterized as being a balloon-expendable stent or a self-expanding stent, depending upon how deployment is affected. Self-expanding stents and balloon-expanding stents differ in many respects and have very different mechanical and dynamic properties. Various technical papers have discussed these differences in detail, and are generally known to those of skill in the art.

Balloon expanding stents are manufactured in the crimped state and are expanded to the vessel's diameter by inflating a balloon, thus plastically deforming the stent. In contrast, self-expanding stents are manufactured at the vessel diameter (or slightly above) and are crimped and constrained to the smaller diameter until the intended delivery site is reached, where the constraint is removed and the stent deployed. Accordingly, balloon expanding stents resist the balloon expansion process, whereas self-expanding stents assist vessel expansion. In other words, while self-expanding stents typically become part of the anatomy and act in harmony with native vessels, balloon-expanding stents change the geometry and properties of the anatomy. From the applicant's perspective, self-expanding stents assist, while balloon-expanding stents dictate.

While balloon-expandable stents generally reach their maximum diameter at the time of deployment (depending on the pressure of inflation), self-expanding stents typically continue to expand post deployment, reaching their maximum diameter several days to weeks later post deployment. With balloon-expandable stents, the vessel should be overdilated (e.g., +10-20%) to overcome the artery's “elastic recoil” (e.g., the tendency for the artery after dilation to return back to its original diameter). The overdilation is necessary to achieve a good apposition of the balloon-expandable stent. Without subsequent balloon dilatation, a balloon-expanding stent may become smaller in diameter over time (chronic recoil). Furthermore, the lesion should be pre-dilated before placement of the balloon-expanding stent because these stents inherently lack any expansile force. In addition, the placement of these stents frequently requires post-placement dilation to achieve optimal apposition of the stent to the vessel wall. Sometimes all of these steps (e.g., pre, during, and post deployment) consist of multiple balloon inflations. These multiple overdilations of the vessel is evidently much more injurious than the damage a self-expanding stent can cause during placement. A properly over-sized self-expanding stent, however, continues to apply a force acting to expand the vessel. Further, a self-expanding stent typically undergoes a negative chronic recoil (that is, a luminal gain), which means that a self-expanding stent continues to open over time, often remodeling the vessel profile. In general, self-expanding stents generally reside near and scaffold the outside of the vessel wall, while balloon-expanding stents remain near the lumen. The negative recoil of self-expanding stents may become an important advantage over drug eluting balloon expanding stents, in which the lumen may actually increase shortly after the deployment by inhibiting the neointima formation (new inner layer of the vessel), but this cell inhibition may result in a patchy covering layer over the stent leaving the balloon-expanding stent exposed to the flow of blood and resulting in in-stent restenosis in the long run.

Radial strength describes the external pressure that a stent is able to withstand without incurring clinically significant damage to the vessel lumen. Balloon-expanding stents can collapse if a critical external pressure is exceeded, potentially having serious clinical implications (stent crush resulting in obstruction of the lumen). On the other hand, self-expanding stents generally have no strength limitation and elastically recover even after complete flattening or radial crushing. Thus, self-expanding stents are ideally suited to superficial locations, such as the carotid and femoral arteries.

Stiffness is defined as how much the diameter of a stent is reduced by the application of external pressure. Axial stiffness is directly reflected in bending compliance. A balloon-expanding stent is typically stiffer than a self-expanding stent of identical design because of the lower elastic modulus of nitinol. Self-expanding stents are much more compliant than balloon-expanding stents of identical design, which is applicable for both delivery and deployment. Self-expanding stents typically adapt their shape to that of the vessel rather than force the vessel to the shape of the stent. Forcing a vessel into an unnatural shape, even if straight and aesthetically pleasing, can lead to high contact forces at the ends of the stent. Flexible links that are plastically deformed during bending accumulate damage and can fracture quickly as a result of fatigue.

Acute recoil refers to the reduction in diameter immediately observed upon deflation of a balloon. A balloon-expanding stent recoils after balloon deflation, whereas self-expanding stents assist balloon inflation (if needed post deployment) and thus there is no recoil of a self-expanding stent. As applied to a vessel, however, both devices generally recoil due to the spring back forces of the vessel and constrictive forces of a significant stenosis.

The delivery profile of a balloon-expanding stent is typically dictated by the profile of the balloon upon which it is mounted. In contrast, self-expanding stent profiles are typically dictated by the strut dimensions (e.g., the width) required to achieve the desired mechanical performance. Current minimum profiles of the two types are very similar, but self-expanding stents have the greater potential to reduce in size. This is expected to play an important role in neurovascular stenting, where both delivery profile and flexibility are essential.

While bending, crushing, and stretching fatigue considerations is often ignored for stents, these factors can be very important in certain indications. One extreme case is the femoral-popliteal artery, but such issues can also be important in coronary vessels because of the systolic expansion of the heart (thereby stretching the stent). For example, older generations of coronary stents were very rigid in the axial direction and not subject to axial fatigue; newer more flexible generations, however, can experience axial deformations and may be prone to fatigue damage. Nitinol performs far better than any other known metal in displacement-controlled environments such as these, which ultimately may mean that this more fatigue-resistant metal offers further advantages under various circumstances.

Newer stent platforms (both balloon and self-expanding) have been designed to increase flexibility, radial strength, torsion, and lengthening or shortening of the vessel, with decreased rates of stent fracture and restenosis. For example, newer-generation self-expanding nitinol stents have a superelastic metallic alloy of nickel and titanium and thinner struts, thereby resulting in better deliverability and less in-stent restenosis.

Percutaneous transluminal coronary angioplasty with stenting has now become one of the cornerstones of treatment for coronary artery disease (CAD). Self-expanding stents and balloon-expandable stents are routinely used in peripheral arterial disease, although balloon-expandable stents have become the stent of choice for coronary arteries. The use of third-generation drug-eluting stents (DES) is currently the preferred method of treatment for all patients with coronary artery disease.

In general, most conventional techniques for treating bifurcation lesions use a balloon expandable stent. This is no surprise, as there is an existing bias in the coronary stent market that favors third generation DES balloon expanding stents over currently available self-expanding versions. For example, one reason that self-expanding stents are not favored is that the majority of these self-expanding stents are nitinol slotted tube designs that are plagued by undesirable features such as having a weak expansile force, are crash-prone, easily break, and are not flexible enough, etc. As another example, balloon-expanding stents have traditionally provided more optimal placement within the vessel, and traditional balloon-expanding stents have provided less recoil than self-expanding stents when placed in particular locations.

However, the deployment of the balloon expanding stents are overly injurious and requires a series of overdilation of the vessel. Further, while the DES coating used with these balloon expanding systems aims to control the reactive abundant neointima formation by inhibiting the growth of the endothelial cells (which works for a short time), the patchy and partial endothelization may result in frequent in-stent stenosis and thrombosis in the long run. For example, the stent wires may not be completely covered, and the bare wires may be a source of thrombus formation (e.g., in-stent restenosis). Another negative aspect of using DES is that while the Everolimus drug inhibits growth factor-stimulated cell proliferation leading to inhibition of cell metabolism, growth, and proliferation by arresting the cell cycle at the late G1 stage, it can sensitize patients and also the use of such drug-treated stent is contraindicated in patients with any immune compromise. The amount of drug, which is released from the stent's polymer coating is said to has clinically insignificant systemic dose but still it has some potential to cause some deleterious effect in some individuals. Further, while the balloon expandable stents in general have greater outward forces after placement than a typical self-expanding stent, their ability to withstand external forces are very limited and they suffer with crush-prone designs. For example, the slotted tube nitinol balloon expanding stents are inherently brittle and breakable and are not resistant to fatigue. Further, how these balloon expandable stents are designed (such as to be flexible) further increase the possibility of fatigue (by thin connecting struts as bridges).

Stents are necessary for both the arterial system and the venous system. Relative to the arterial system, the venous system is characterized by low pressure, low velocity, large volume, and low resistance. The heart, pressure gradients, the peripheral venous pump, and competent valves interact together to overcome the hydrostatic pressure induced by gravity. The larger veins serve as the primary capacitance vessels where most of the blood volume is found and where regional blood volume is regulated. Venous stenosis is intimal hyperplasia and fibrosis causing progressive vessel narrowing and outflow obstruction. Venous stenosis most commonly affects the axillary, brachial, cephalic, or brachiocephalic veins of the upper extremities, or the superior vena cava, but can also affect the central veins in the abdomen and the pulmonary artery and veins. Common causes are placement of central venous catheters, pacemaker leads, hemodialysis catheters, prior radiation, trauma, or extrinsic compression. The use of venous stents is a medical necessity in patients with disabling or life-threatening occlusive or stenotic disease of the central veins that extend from the iliofemoral veins to the subclavian veins.

There are numerus prior art stenting applications for venous stenting. The most extensive clinical experience within venous stenting has been off-label use of the Wallstent (available from Boston Scientific). The Wallstent is available in large diameters and has an Elgiloy (similar to stainless steel) braided construction that provides flexibility. Unfortunately, because of the flexibility of the Wallstent, when constricted, its length varies resulting in decreased deployment accuracy. Accurate deployment is also limited by foreshortening of up to 40%. If there is compression near the end of the stent, as is often the case near the ilio-caval junction, the Wallstent may form a narrowed cone shape, which decreases flow, or the stent can migrate as it is squeezed away by a compressive lesion. Another pitfall of the Wallstent is in the setting of bilateral iliac stenting. In order to decrease problems with jailing of the contralateral side or narrowing of bilateral stents in the inferior vena cava, multiple techniques have been developed including the double barrel, fenestration, and Z stent techniques. Unfortunately, none of these techniques are ideal and may result in the need for reintervention.

The prior art also includes dedicated venous stents, many of which comprise self-expanding nitinol stents. For example, the Cook Zilver Vena venous stent has an open cell design and is available in 14-16 mm diameters and 60-140 mm lengths. The stent has a 7-French platform that is compatible with 0.035″ wires. The open cell design affords flexibility and the stent has minimal foreshortening. The Veniti Vici Venous stent, distributed by Boston Scientific, has a closed cell design with uniform end-to-end shape and strength. It is available in 12-16 mm diameters and 60-120 mm lengths. It has a 9-French platform compatible with 0.035″ wires. The closed cell design and sinusoidal strut rings give strength while the alternating curved bridges afford flexibility. It is a strong stent, with a sufficient surface area, performing well in May-Thurner syndrome patients, with good stent integrity. The Optimed sinus-Venous stent has a hybrid design trying to balance the need for both radial force and flexibility. The stent comes in 10-18 mm diameters and 60-150 lengths with a 10-French platform. With the sinus-Venous device, one has to be very accurate with the initial deployment because there is no change of repositioning. It is a flexible stent and with the correct deployment technique it also has a high crush resistance. The sinus-XL Flex stent is easier to deploy, though it is less flexible, and it has a tendency to kink at the flex points. The Optimed sinus-XL stent has a closed design affording high radial force. The stent comes in 16-36 mm diameters and 30-100 mm lengths with a 10-French platform. The sinus-XL in intended for large linear vessels including the aorta and the inferior vena cava. An Optimed sinus-XL 6 F also has been developed with diameters of 14 and 16 mm. Another stent, the Optimend sinus-XL Flex comes in large diameters (14-24 mm) but has an open cell design to afford it more flexibility. It comes in 40-160 mm lengths and also has a 10-French platform. The Optimed sinus-Obliquus has a hybrid design with a closed cell design oblique-shaped central end, an open cell design mid-segment, and an anchor ring at the peripheral end. The closed cell oblique segment allows for increased radial force and crush resistance at the iliocaval stress point while minimizing overlap of the contralateral common iliac vein. The open cell design of the mid-segment provides flexibility and conformity to the stent. The peripheral end anchor helps with stent fixation. The sinus-Obliquus stent, with its 10-Fr platform, comes in 14- and 16-mm diameters and 80-150 mm lengths. Medtronic has more recently initiated a dedicated venous stent investigational device exemption study, named the ABRE IDE study. The Abre venous self-expanding stent has an open cell design with 3 points of connection between cells to afford it flexibility and conformity. This stent system has a triaxial shaft design to aide with delivery. This device is delivered through a 9-Fr system and will be available in diameters up to 20 mm.

Despite the recent advance in the venous stenting field, still no ideal venous stent exists. The majority of the recently developed venous stents designs lacks several important features of a desirable venous stent. For example, most of the conventional venous stents are nitinol slotted tube designs that inherently lack adequate radial forces, equipped with connective struts to facilitate flexibility but these elements simultaneously make the structure vulnerable to outer forces, bringing in factors of long-term fatigue, etc. There is a real need to develop stents that feature the most requirements for an ideal venous stent.

Several self-expanding devices are described in U.S. Pat. No. 7,018,401 (“the '401 Patent”) and Pat. No. 8,739,382 (“the '382 Patent”), each incorporated herein by reference. Each of the '401 and '382 Patents provides a summary of some of the prior art relevant to the present disclosure, the prior art which is incorporated herein by reference.

The '401 Patent is directed to a self-expandable, woven intravascular device for use as stents, filters, and occluders for insertion and implantation into a variety of anatomical structures. The devices may be formed from shape memory metals such as nitinol, may be formed from a single wire, and may be formed by either hand or machine weaving. The devices may be created by bending shape memory wires around tabs projecting from a template and weaving the ends of the wires to create the body of the device such that the wires cross each other to form a plurality of angles, at least one of the angles being obtuse. In general, the proximal end of the straight stent is created by back-weaving the wire strands and then welding them at the appropriate points. As one example, FIGS. 1A, 1B, and 1C of the '401 Patent illustrate examples of a prior art stent, which are reproduced in the current disclosure as FIGS. 1A, 1B, and 1C, respectively. As reproduced from the '401 Patent at col. 15, lines 20-34 (and incorporated herein by reference):
“Body 10 is both radially and axially expandable. Body 10 includes front or distal end 12 and rear or proximal end 2. As shown in FIG. 1A, end 12 has a plurality of closed structures. These closed structures may be small closed loops 6 or bends 8 (FIG. 1B). Both bends 8 and small closed loops 6 may be formed by bending a wire 5 at a selected point located between the ends 7 of wire 5 (FIG. 1C shows small closed loops 6). For most applications, the selected point of the bend or small closed loop may be close to the midpoint of wire 5, as shown in FIG. 1C with respect to small closed loop 6. FIG. 1C also shows both ends of wire 5 being located proximate end 2 of body 10 (although the remainder of body 10 is not shown). Body 10 is formed by plain weaving wires 5 . . . .”

The '382 Patent is directed to a woven, self-expanding stent device that has one or more strands and is configured for insertion into an anatomical structure. The device includes a coupling structure (that is not a strand of the device) that secures two different strand end portions that are substantially aligned with each other. As one example, FIGS. 1, 2, and 3 of the '382 Patent illustrate examples of a prior art stent and formation thereof. FIG. 1 of the '382 Patent shows an example of device 100 that has one or more strands and is configured for insertion into an anatomical structure. Device 100 (which is a stent) is created woven according to techniques disclosed in the '401 Patent, and in particular from six strands (wires) that possess twelve strand halves 10. There are no free strand ends at the end of device 100. As shown in FIG. 4 of the '382 Patent, each half strand was secured to only one other half strand, which either belonged to the same or a different strand. After heat treatment, the device can be immediately quenched in deionized water until cool. Next, the free strand ends of the device can be back-braided as desired, baked, and then immediately quenched in deionized water until cool. FIG. 3 of the '382 Patent shows device 100 after half of the twelve loose strand ends have been back-braided. The coupling structures that are used (for stents, the number of coupling structures will preferably equal the number of strands) may be axially aligned with the strands, such as those coupling structures displayed in FIGS. 3, 4A, 4B, 6, and 7 of the '382 Patent, incorporated herein by reference.

Abbot offers a stenting system known as Supera. The instructions for use of the Supera Stent is publicly available and known to those of skill in the art and is incorporated herein by reference. The Supera Peripheral Stent System consists of a closed end, braided self-expanding stent made of nitinol (nickel-titanium alloy) wire material that is pre-mounted on a 6Fr delivery system. The stent typically does not include radiopaque markers, but to increase the radiopacity of the stent, the wire strands can be nitinol microtubings with a platinum core.

The Supera stent is a platform stent. In other words, the Supera structure allows the design of any size and/or length stent that is needed for the different vascular and non-vascular regions of the body, including the coronary artery. In general, any structure can be made from the appropriate number and size of wires to create a mesh that fits best given a particular anatomy's needs. For example, for coronary application, a Supera stent can be produced with a smaller number of wires than traditionally used: instead of twelve wires (six pairs), the stent may use six, eight, or ten wires to form the mesh. The mesh tightness can be adjusted according to the specific need, from open cell design to very tight mesh where the wire strands obtuse angles approaching 180 degrees. If a stent is made with a mesh size that is looser than the original design (that is the obtuse angles in the mesh are reduced), that will be the stent nominal diameter that is imprinted to the wires by the heat treatment. Such a stent may still feature a significant radial force what it is deployed with in nominal diameter.

The Supera stent sizes are labeled based on the outer stent diameter. A stent should initially be chosen such that its labeled diameter matches the reference vessel diameter (RVD) proximal and distal to the lesion. Final stent selection should be confirmed after lesion pre-dilation: if possible, the stent diameter should match the prepared lesion diameter 1:1. Due to the mechanical behavior of the woven Supera stent, the stent should not be oversized by more than 1 mm relative to the RVD. This ensures optimum deployment of the Supera stent, maximizing radial strength, and assisting in accurate stent length deployment. Choosing a labeled diameter to match the reference vessel diameter, then appropriately preparing the vessel to match that stent's diameter will result in a stent that is properly sized to the vessel. The vessel should be prepared utilizing standard angioplasty technique using a balloon size greater than or equal to the stent diameter. The post-dilated vessel should be at least the size of the stent diameter.

The Supera stent attempts to mimic the natural structure and movement of the relevant anatomy. The interwoven nitinol design creates a stent that supports rather than resists the vessel. By pre-dilating and matching the stent and vessel 1:1, the Supera stent supports the vessel with minimal chronic outward force. The Supera stent has increased strength and flexibility, with more than four times the compression resistance of typical standard slotted tube design nitinol stents. In severely calcified lesions, the Supera stent has visible compression resistance, maintaining a round, open lumen for normal, healthy blood flow in challenging anatomy.

The over-the-wire stent delivery system for Supera is compatible with a 0.014″ and a 0.018″ guide wire and comes in lengths of 80 cm and 120 cm (6Fr). The delivery system includes a reciprocating mechanism (e.g., stent driver) that incrementally moves the stent distally out of the outer sheath. This motion allows for the distal end of the stent to first come in contact with the targeted vessel, setting the distal reference point, and continues to feed the stent out of the sheath as the target wall is exposed by the proximal movement of the catheter. This stent deployment is achieved by the reciprocation of the thumb slide located on the handle. On the final stroke, the deployment lock is toggled, and the last deployment stroke is made.

A Supera based stent has a strong expansile (outward) force that can be easily adjusted by carefully selecting the size and the number of the wires for the mesh. The stent structure allows to create stents between a relative open cell arrangement and a very tight mesh size. The stent is biomimetic, that is, it can easily follow and accommodate to even the most tortuous anatomy while its inner diameter is never compromised. The Supera stent has perfect conformability. The Supera stent can withstand repeated pulsatile outer compressing forces, as well as axial movements (shortening/elongation) which is a factor in the coronary arteries on the surface of the ever-pulsating heart. The Supera stent is known to be able to endure very strong complex forces in the most challenging anatomy, namely in the femoropopliteal region. It can withstand compressive forces (popliteal artery), torque, shortening and elongation (femoral artery) even when these forces are present in combination. The stent may allow to eliminate the need for pre- and also post placement dilation, therefore its placement is much-much less injurious that that of the balloon expandable stent.

The Supera stent offers numerous benefits over other self-expanding stents and in particular balloon-expanding stents. In particular, Supera based coronary stents can overcome many of the inherent problems of balloon-expandable stents, such as high-pressure balloon inflations, overexpansion of a narrow segment distal to the stent, increasing the risk of an edge dissection, and underexpansion of a proximal segment resulting in poor stent apposition. Further, Supera based coronary stents eliminate the need for multiple very injurious balloon dilations and makes unnecessary the use of a DES coating that (while it has been proven to be advantageous to restrain neointima formation and increase lumen gain in the early phase of post-stenting) may result in patchy coverage of wire struts by neointima that can negatively influence in-stent restenosis in the long run. Self-expanding stents in general, and the woven nitinol stent in particular, embed deeply in the vessel wall reaching the muscular layer. As a result, the neointimal coverage can be complete. That is important to avoid in-stent restenosis later. Even a larger layer of the neointima will be adequately compensated by the fact that the stent making lumen gain after deployment for days or weeks. Still further, because balloon expandable stents cannot be used in vessels with a diameter larger than 4.5 mm, these lesions cannot be treated with them. The Supera based coronary stent's versatility in diameter and length allows treatment over a wide range of lesion sizes and configurations.

Conventional stents—whether balloon assisted or self-expanding—possess certain shortcomings that inhibit their ease and range of use. In particular, the dominant use of a balloon expandable stent in the coronary field is problematic, particularly in view that some self-expanding stents have several advantageous features over the balloon expanding counterpart. Self-expanding stents in general, and in particular a Supera based woven nitinol stent, provides several characteristics that make these stents more advantageous for the coronary application than the balloon expandable stents.

Despite the apparent benefits of a Supera based stenting system, it fails to address several problems and several characteristics of the different anatomical applications. The Supera stent is designed to be a robust stent, with excellent conformability, flexibility and crush resistance, which are the main requirements for a femoropopliteal stent. The femoropopliteal anatomy is the most challenging anatomy from a mechanical point of view. Because of the ambulation the femoropopliteal artery is exposed to extremely harsh and combined forces. These forces, which are repetitive in nature, are elongation/shortening, compression, torsion, and flexion, acting alone and/or in combination. The structure of the stent should be able to follow these movements and accommodate to them. These phenomena, which are aptly described as Supera's biomimetic characteristics, serve these purposes extremely well.

The woven stent design, however, is a platform stent, meaning that the basic structure of the stent allows for creating numerous different versions of the stent that are much more suitable to meet the special requirements of completely different anatomical locations. For example, for a coronary artery the robustness of the femoropopliteal Supera stent is not needed. Nevertheless, there are repetitive pulsating forces (on average 72/min) on the stents in that location that are very significant in the long run and necessitates a fatigue-free stent that does not break. Further, its radial force may be less with still excellent flexibility and conformability and these requirements can meet by structural modifications of the basic stent structure.

The use of an adequately modified woven stent for venous lesions may be another primary example. The arterial and venous anatomy are completely different structurally and hemodynamically, therefore the stents used in the arterial side are not able to meet the requirements of the venous locations. For example, the veins have much thinner walls with practically no muscle layer in it. A robust stent with high radial force would be completely inadequate in these locations; these stents would make the venous segments tube-like, rigid with undesired hemodynamic alterations. In addition, the venous wall would be exposed to extremely high forces that would be detrimental. In short, the woven stent design allows for numerous structural modifications that help find the stent with optimal characteristics for each of the different anatomical regions.

The statements in this section are intended to provide background information related to the invention disclosed and claimed herein. Such information may or may not constitute prior art. It will be appreciated from the foregoing, however, that there remains a need for an improved method, device, and system for treating ostial lesions and branching anatomies, particularly with self-expanding stents. A need exists for an improved method, device, and system for treating complex lesions including branching and bifurcated anatomies in the arterial system as well as the venous system. A need exists for a self-expanding stent and stent system that addresses problems such as high-level injuries of the balloon expandable stents, branching/bifurcated lesions, ostial lesions, and lesions with complex anatomy. A need exists for an improved self-expanding stent that provides for increased structural modifications and the ability to be used in a wide variety of applications. Such disadvantages and others inherent in the prior art are addressed by various aspects and embodiments of the subject invention.

### SUMMARY OF THE INVENTION

This disclosure provides a self-expandable, woven intravascular device for use as a stent (both straight and tapered) in a variety of anatomical structures, including vessels in the arterial and/or venous system. The stent provides enhanced structural modifications to conventional woven stents by varying the wire(s) utilized in the woven stent. The stent may be formed from shape memory metals such as nitinol. The stent may utilize multiple, parallel wires instead of a single wire, an increased number of wire pairs and/or pins to vary the mesh size, and/or a bundle of wires (e.g., a plurality of individual wires coupled together) instead of a single wire. The individual wires may each be twisted to provide an overall twist to the wire bundle. The bundle of wires may comprise different sized wires and/or wires of different materials.

Disclosed is a self-expanding woven stent, comprising a plurality of shape memory wires woven together to form a body having a first end and a second end, wherein the plurality of shape memory wires comprises a first wire and a second wire, wherein the first wire is arranged substantially parallel to the second wire. Each of the wires may be substantially adjacent to each other, and may each comprise a wire bundle composed of a plurality of individual, smaller wires. Each of the plurality of shape memory wires may bend at a plurality of points creating a mesh of substantially perpendicular crossing wires that form a plurality of cells and a plurality of angles. The mesh formed by the crossing wires may be an open celled mesh or a closed cell mesh, thereby created by different weave densities of the crossing wires. In one embodiment, each of the plurality of angles are approximately 90 degrees, while in other embodiments the angles may comprise acute angles or obtuse angles.

The stent may have a flanged portion and a body that is tubular, substantially straight, and/or substantially tapered. The stent may be an arterial stent or a venous stent. The stent may be configured for deployment in at least one of the following arteries: coronary, iliac, femoropopliteal, infrapopliteal, carotid, vertebral, subclavian, and intracranial. The stent may be configured for deployment in at least one of the following veins: inferior vena cava, a superior vena cava, axillary, brachial, iliac, and femoral.

Disclosed is a self-expanding woven stent, comprising a body with a first end and a second end, wherein the body comprises at least one shape memory wire woven together, wherein the at least one shape memory wire crosses at a plurality of positions to form a plurality of cells and a plurality of angles, wherein the plurality of angles are approximately 90 degrees.

Disclosed is a self-expanding woven stent, comprising a body with a first end and a second end, wherein the body comprises at least one shape memory wire woven together, wherein the at least one shape memory wire comprises a wired bundle, wherein the at least one shape memory wire crosses at a plurality of positions to form a plurality of cells and a plurality of angles.

The wire bundle may comprise a plurality of individual wire strands, and they may be twisted together to form a generally helical wire. Each of the wire strands may be approximately the same diameter, or they may have different diameters. Each of the wire strands may have the same material or composition, or may be formed of different wire compositions. For example, the at least one wire bundle may have one or more microtubings with a platinum core. The wire bundle may have compressed wires, non-symmetrical wires, substantially compacted wires, and/or substantially round wires. The at least one wire bundle may comprise any number of individual wires, including at least three, at least seven, and at least 19 individual wires (or individual wire bundles).

The at least one wire bundle may comprise a plurality of individual wire segments, wherein at least one of the plurality of individual wire segments comprises an inner wire bundle. The at least one wire bundle may comprise a plurality of inner wire bundles, wherein each of the inner wire bundles comprises a plurality of individual wires. The at least one wire bundle may comprise a first plurality of wires with a first diameter and a second plurality of wires with a second diameter. The at least one wire bundle may comprise an inner set of wire bundles and an outer set of wire bundles. In one embodiment, the inner set of wire bundles may comprise a first set of wires with a first diameter and a second set of wires with a second diameter. In one embodiment, the outer set of wire bundles may comprise a first set of wires with a first diameter and a second set of wires with a second diameter.

Each of the individual wires of the stent may itself comprise a wire bundle formed of individual wire segments. Further, the stent may comprise a plurality of wires, and some or all of these may be wire bundles. For example, the at least one shape memory wire may comprise a plurality of shape memory wires, wherein each of the plurality of shape memory wires comprises a wire bundle to form a plurality of wire bundles for the stent. Each of these plurality of wire bundles may be substantially parallel and/or adjacent to each other. In one embodiment, each of the plurality of wire bundles comprises an inner set of wire bundles and an outer set of wire bundles. In another embodiment, the plurality of wire bundles comprises a first plurality of wire bundles with a first diameter and a second plurality of wire bundles with a second diameter.

Disclosed is a method for forming a self-expanding stent, the method comprising forming a stent by bending at least one shape memory wire at a first plurality of bends to form a mesh, wherein each of the plurality of bends is approximately 90 degrees and heat treating the at least one shape memory wire.

Disclosed is a method for forming a self-expanding stent, the method comprising forming a stent by weaving a plurality of shape memory wires together to form a body with a first end and a second end, wherein the plurality of shape memory wires is substantially parallel to each other. In one embodiment, the plurality of shape memory wires may be substantially adjacent to each other. The plurality of shape memory wires may comprise at least three wires. In other embodiments, the plurality of shape memory wires may comprise a plurality of wire bundles. Each of the plurality of shape memory wires may cross each other to form a plurality of cells and a plurality of angles. The angles may be approximately 90 degrees, may be acute angles, or be obtuse angles. In one embodiment, the method may further comprise bending the plurality of shape memory wires around a plurality of protrusions on a template.

In one embodiment, the disclosed stent is formed around a template with a plurality of protrusions. There may be a first plurality of pins at a first position and a second plurality of pins at a second position. The positions may be relative to each other, a radial position, and/or a longitudinal position. In one embodiment, the first plurality of pins is located at a first circumferential position on a template and the second plurality of pins is located at a second circumferential position on the template. In another embodiment, the second plurality of pins is located proximally on the template to the first plurality of pins. In one embodiment, the amount of the second plurality of pins is approximately half of the amount of the first plurality of pins, whereas in other embodiments the number of the second plurality of pins is greater than the first plurality of pins. In one embodiment, the second plurality of pins is substantially in-line with the first plurality of pins, while in another embodiment at least some of the second plurality of pins is located at an off position relative to the first plurality of pins. In one embodiment, the plurality of protrusions comprises a first plurality of pins located at a first circumferential position, a second plurality of pins located at a second circumferential position, and a third plurality of pins located at a third circumferential position.

Disclosed is a method for forming a self-expanding stent, the method comprising forming a stent by weaving at least one wire bundle together to form a body with a first end and a second end, wherein the at least one wire bundle crosses at a plurality of positions to form a plurality of cells and a plurality of angles. In one embodiment, the at least one wire bundle comprises a plurality of wire bundles. In one embodiment, the plurality of wire bundles is substantially parallel to each other and/or adjacent to each other. In one embodiment, the method further comprises twisting the at least one wire bundle during the weaving step. In another embodiment, the method comprises twisting a plurality of wire segments of the wire bundle together to form the at least one wire bundle.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A, 1B, and 1C are reproduced from FIGS. 1A, 1B, and 1C, respectively, of U.S. Pat. No. 7,018,401.

FIGS. 1D, 1E, and IF are reproduced from FIGS. 1, 2, and 3, respectively, of U.S. Pat. No. 8,739,382.

FIG. 2A illustrates one schematic of a stent according to one embodiment of the present disclosure.

FIG. 2B illustrates one schematic of a stent according to one embodiment of the present disclosure.

FIG. 3A illustrates one schematic of a wire mesh pattern of the stent from FIG. 2B made from two parallel wires, according to one embodiment of the present disclosure.

FIGS. 3B and 3C illustrates various schematics of a wire connecting piece for a stent, according to one embodiment of the present disclosure.

FIG. 4A illustrates one schematic of a stent according to one embodiment of the present disclosure.

FIG. 4B illustrates one schematic of a wire mesh pattern of the stent from FIG. 4A made from three parallel wires, according to one embodiment of the present disclosure.

FIG. 5 illustrates one schematic of a stent according to one embodiment of the present disclosure.

FIG. 6 illustrates one schematic of a stent according to one embodiment of the present disclosure.

FIG. 7A illustrates one schematic of a wire mesh pattern of a stent according to one embodiment of the present disclosure.

FIG. 7B illustrates one schematic of a wire mesh pattern of a stent according to one embodiment of the present disclosure.

FIGS. 8A-8D illustrate various schematics of wire bundles according to one embodiment of the present disclosure.

FIGS. 9A-9D illustrate various schematics of wire bundles according to one embodiment of the present disclosure.

FIGS. 10A and 10B illustrate various schematics of wire bundles according to one embodiment of the present disclosure.

FIGS. 11A and 11B illustrate various schematics of a twisted wire bundle according to one embodiment of the present disclosure.

FIGS. 12A-12C illustrate various schematics of wire bundles according to one embodiment of the present disclosure.

FIG. 13 illustrates one schematic of a wire mesh according to one embodiment of the present disclosure.

FIGS. 14A-14D illustrate various schematics of wire meshes according to one embodiment of the present disclosure.

FIGS. 15A-15D illustrate various schematics of wire meshes according to one embodiment of the present disclosure, wherein the mesh uses three rows of wires.

FIGS. 16A-16K illustrate various schematics of wire meshes according to one embodiment of the present disclosure, wherein the mesh uses two rows of wires.

### DETAILED DESCRIPTION

Various features and advantageous details are explained more fully with reference to the nonlimiting embodiments that are illustrated in the accompanying drawings and detailed in the following description. Descriptions of well-known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure. The following detailed description does not limit the invention.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures, or characteristics may be combined in any suitable manner in one or more embodiments.

### Overview

In one embodiment, disclosed is a self-expanding woven stent that provides enhanced structural modifications to conventional woven stents, such as a Supera based stent. In one embodiment, the disclosed stent varies the configuration of the wire(s) utilized in the woven stent to vary the characteristics of the stent. In one embodiment, the disclosed stent utilizes multiple, parallel wires instead of a single wire. In one embodiment, the disclosed stent utilizes an increased number of wire pairs and/or pins to vary the mesh size. In one embodiment, the disclosed stent utilizes a bundle of wires (e.g., a plurality of individual wires coupled together) instead of a single wire. In one embodiment, the individual wires may each be twisted to provide an overall twist to the wire bundle. In one embodiment, the bundle of wires may comprise different sized wires and/or wires of different materials. In one embodiment, some of the utilized wires may be micro-tubes filled with a radiopaque material, such as platinum. In one embodiment, the disclosed stent may be formed at least partially of biodegradable materials and may comprise a bioresorbable material.

The individual wires used in the stent may be metallic or non-metallic wire, which may include one or more twisted or woven filaments. The wire can take many different shapes, sizes, materials, and can have differential compositions and characteristics. For example, the wire may be round, flat, rectangular, oval, triangular, serrated, and other configurations, and may be symmetrical or non-symmetrical (e.g., compacted). In other words, a wire is not necessarily round or symmetrical. Similarly, the wire may be tubular or solid, as well as cored or woven. The material, shape, and diameter of the wire is variable based upon various desired characteristics of the stent and the intended stent application.

The disclosed stent provides numerous benefits. For example, the proposed wide scale of modifications for the disclosed stent make it possible to find the best mechanical characteristics for a dedicated anatomical location. The disclosed stent allows for a wide range of stent optimization that provides the best possible acceptance, integration, and/or incorporation of the stent as a foreign body by the living structure. While the general characteristics of the woven design such as flexibility, conformability, and/or crush resistance will remain, they may be fine-tuned (optimized) with properly selected radial force, adequate mesh tightness (closed cell vs. open cell designs), and fatigue-freeness, among other capabilities.

In one embodiment, the disclosed stent varies the type of utilized wire(s) of the stent, as the wire caliber has a significant impact on the strength of the stent and other properties (such as radial/expansile force, crush resistance, and hoop strength). For example, if the size of the nitinol wire is increased, different characteristics of the stent may proportionally increase. As another example, if more wires are utilized in the stent structure, the overall coverage of the stent increases as well as the increase of the angles of the crossing wires resulting in a tighter wire mesh (also resulting in an overall increase in strength). For some applications, the stent coverage is very important, as it dictates whether an open cell or closed cell stent design is used, as well as whether the stent is suitable for a particular application (e.g., in a carotid artery application or in the venous system). In other applications, certain modifications may result in a desired decrease in strength of the stent to achieve the best match/design between the stent and the given anatomical structure.

In one embodiment, the disclosed self-expanding stent allows it for being used as a platform stent. In other words, the disclosed stent can be used to produce stents that are suitable for use essentially anywhere in the body. The disclosed stent can be used in the vascular system (e.g., arterial and venous stents) or the non-vascular system (e.g., biliary tree, colon, etc.). The disclosed stent also provides for a wide range of sizes, including for large diameter stents (aorta, vena cava) or very small caliber stents such as coronary and intracranial stents. In short, the various anatomical territories with a given body requires different stents that best suit the particular structure and function. The basic structure of the disclosed stent offers several ways to meet the requirements of the various anatomical structures. As another example, the disclosed stent may be used alone or be part of a modular stent assembly in which a plurality of stent portions may be coupled together after placement within an anatomical body. Furthermore, the disclosed stent can be configured with different mesh tightness, radial force, diameter, and length as well as taper, each of which may fit a wide variety of anatomical structures all over the body. In one embodiment, the variability in the mesh tightness, the number, and the size of the wires used to create the mesh offers limitless variations that serves the goal to produce the ideal stent for various applications.

In one embodiment, the disclosed stent is a self-expanding stent as opposed to balloon expandable stents typically used in the prior art. Balloon expandable stents require a series of balloon dilation to achieve the desired lumen gain and stent-strut apposition in the vessel. Typically, these balloon inflations are performed before, during, and post deployment of the stent. Not infrequently, each phase uses several balloon inflations. In general, each balloon inflation overextends the vessel wall by at least 10-20 percent to offset the vessel's elastic recoil. The repeat balloon inflations are overtly injurious and as a result the vessels inner layer (endothel) reacts with abundant production of neointima. This thick neointima does not only cover the stent struts but its bulkiness restricts the lumen causing luminal loss (in-stent restenosis). For these reasons, the balloon expanding coronary stents have recently been using with drug eluting systems (DES) to counterbalance the neointima proliferation. Although the antimitotic drugs can efficiently block the neointimal formation and thereby the in-stent restenosis, it may create a patchy intimal layer coverage in the long run, which in turn may lead to a chronic in-stent restenosis with luminal loss. The DES systems are already in their third generation but minimizing the injuriousness on the vessel wall by using self-expanding stents with optimized mechanical characteristic would be the ideal solution.

Self-expanding stents in general, and in particular a Supera-based woven nitinol stent, provides several characteristics that make them more advantageous for coronary applications than prior art balloon expandable stents. For example, the disclosed self-expanding stent has robust redial forces that are even throughout the stent length, has excellent conformability even in the most tortuous vasculature without compromising its lumen, is crush-resistant, and can withstand tens of millions of pulsating forces without a break. In addition, the disclosed stent design allows for creating stents with physical characteristics which are ideal for the given type of vasculature. As another example, deployment of the disclosed self-expanding stent is much less injurious than balloon expandable stents, and there is no need to use balloon inflations to get an appropriate apposition to the vessel wall. As another example, the self-expanding stent has much better conformability and is able to adapt to the given anatomical situation as compared to balloon stents (which generally straighten the stented segments because the balloon itself is not flexible). In particular, the mechanism of post dilation of the balloon expanding and the self-expanding stent is different; whereas the balloon expanding stent requires significant over-dilation of the vessel to achieve the appropriate apposition, the disclosed self-expanding stent inherently withstands any over-dilation, that is, the inflation of the balloon only facilitate the stent to take on its maximum diameter. In one embodiment, if the disclosed stent size is determined precisely according to the diameter of the vessel, any possibility of over-dilation of the self-expanding stent is eliminated. The disclosed self-expanding stent can overcome many of the inherent problems of balloon-expandable stents, such as high-pressure balloon inflations, overexpansion of a narrow segment distal to the stent, increased risk of an edge dissection, and underexpansion of a proximal segment resulting in poor stent apposition. Limitations of self-expanding stents in general include difficulty with precise placement (foreshortening), especially in ostial lesions, and inability to oversize the stent beyond its “set” diameter. These prior art stents, being bulkier, may also be difficult to deliver.

### Multiple Wire Sets

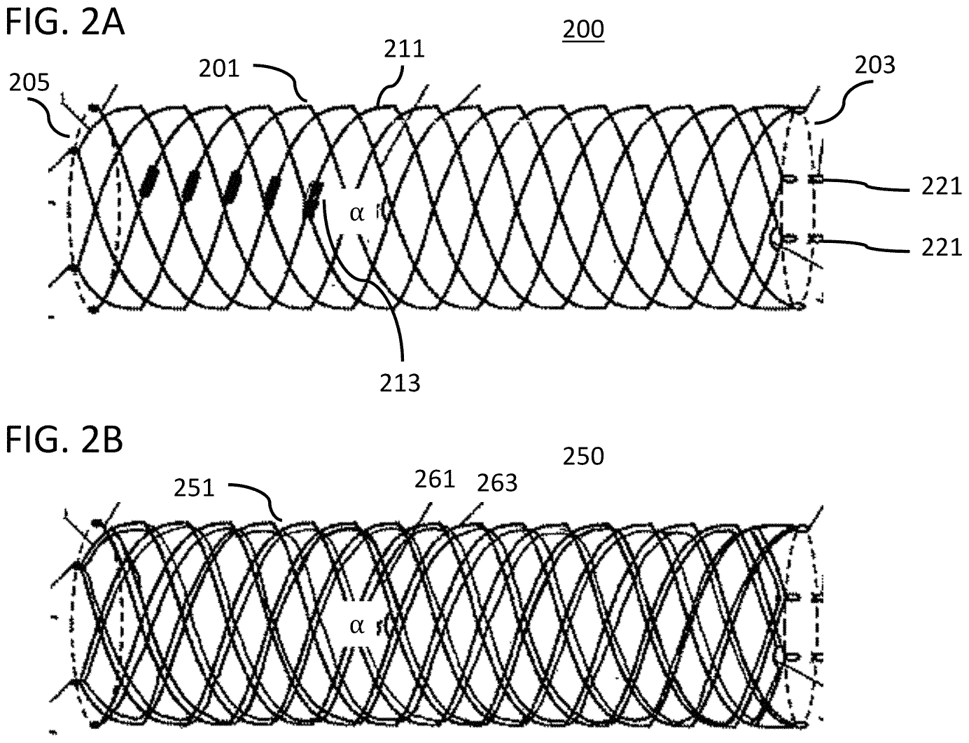

FIG. 2A illustrates a basic structure of a woven stent according to one embodiment of the present disclosure. In one embodiment, stent 200 may be a self-expanding stent and may comprise body 201. Body 201 may be both radially and axially expandable. In one embodiment, the stent may be similar to the self-expanding woven stents disclosed in U.S. Pat. No. 7,018,401 (“the '401 Patent”) and Pat. No. 8,739,382 (“the '382 Patent”), each incorporated herein by reference. In another embodiment, the stent may be similar to the stent commercially known as SUPERA.

Body 201 may have a substantially tubular shape, uniform shape, or a substantially tapered shape, among other configurations. In one embodiment, body 201 may be formed of a plurality of wires 211 that are woven together to form a mesh or grid body for stent 200. A plurality of wires may be used to form the mesh, such as two, four, six, eight, ten, or twelve or more wires. As is known in the art, the size, diameter, and number of wires 211 may be changed to produce the desired configuration and characteristics of stent 200. In one embodiment, the mesh tightness can be adjusted according to the specific need, from open cell design to closed cell design, straight or tapered design, and with or without metallic materials. In one embodiment, the mesh density is variable and/or different between different portions of the stent. For example, a first portion of the stent may be woven with a tighter mesh than a second portion of the stent, that is, the obtuse angles between the wire strands are increased. Alternatively, a particular stent portion can be woven with a looser mesh size, that is the obtuse angles between the wire strands are decreased. These variations in the weave may result in optimal construction of the stent depending on the application and relevant anatomical structure. Thus, the mesh tightness, the radial force, the stiffness and the flexibility of the stent can be set in a very wide range, thereby providing a mesh (and resulting stent structure) offers the ideal stent for the particular anatomy.

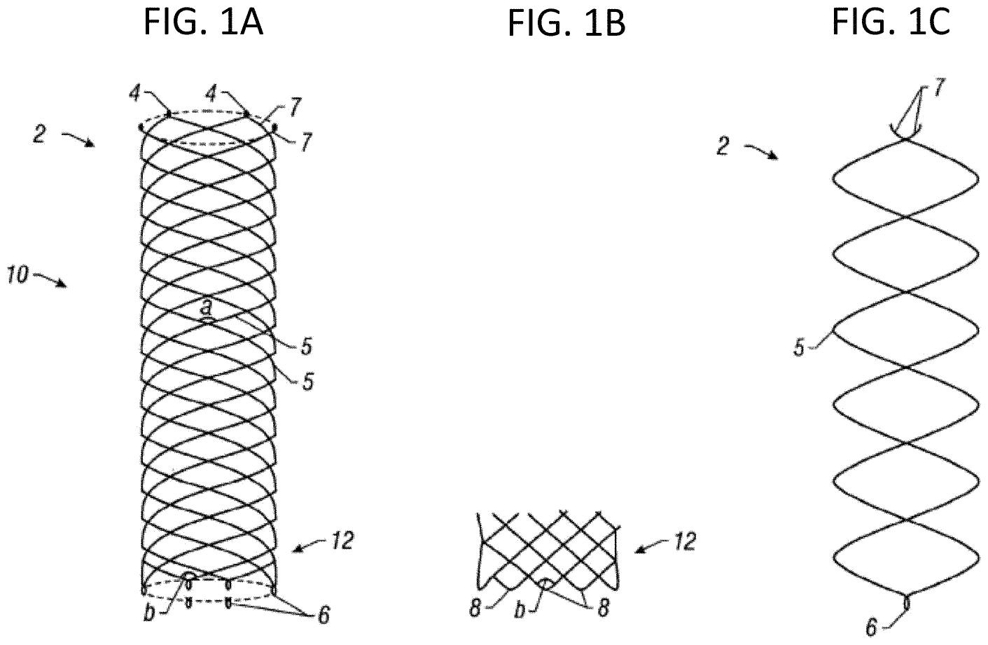

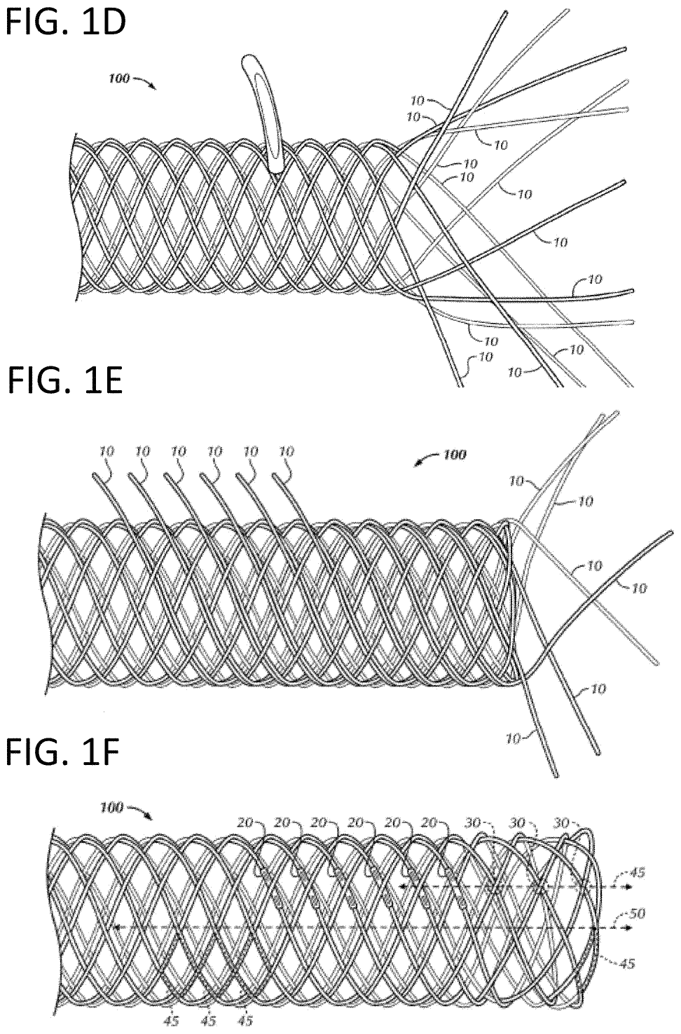

In one embodiment, body 201 comprises leading or distal end 203 and trailing or proximal end 205. Wires 211 may be shape memory (e.g., nitinol or other similar shape memory wire) and be bent approximately at their mid-portion, thereby forming wire pairs. End 203 may have a plurality of closed structures 221. These closed structures may be small closed loops 6 or bends 8 (see FIGS. 1A and 1B). As is known in the art, the bends and small closed loops may be formed by bending wire 211 at a selected point located between the ends of the wire. For many applications, the selected point of the bend or small closed loop may be close to the midpoint of the wire, as shown in FIG. 1C. FIG. 1C also shows both ends of wire 5 being located proximate end 2 of body 10 (although the remainder of body 10 is not shown). In one embodiment, points 221 on the wires are used to hook the stent on pins attached to a cylindrical template for securing the stent body during the weaving process. In one embodiment, trailing end 205 is back woven similarly as seen in FIGS. 1D-1F and the respective wires are united with a connection piece 213 and coupled together via any number of mechanisms, such as crimping, laser welding, etc.

As described in the '401 Patent and the '382 Patent, the disclosed stent may be formed by either hand or machine weaving a plurality of individual wires, filaments, and/or strands together. For example, as is known in the art, the stent may be created by bending shape memory wires around tabs projecting from a cylindrical template, and weaving the ends of the wires to create the body of the device such that the wires cross each other to form a plurality of angles. In general, the proximal end of the straight stent is created by back-weaving the wire strands and then welding them at the appropriate points as seen in FIGS. 1D-1F.

In one embodiment, the self-expanding nature of the disclosed stent allows the stent to expand against the plaque in the artery and exert an outward radial force that resists compression. The greater the density (e.g., the more it is a closed cell stent), the greater radial force the stent exerts on the artery. However, excessive radial force can cause stent impaction and plaque protrusion; the dense mesh in turn will prevent plaque protrusion from occurring. In some embodiments, a portion of the stent is partially or substantially tapered (instead of substantially straight). For example, tapered designs can be useful when deploying stents across the carotid bifurcation, as the common carotid artery (CCA) is typically larger than the internal carotid artery (ICA). The tapered design means that the maximum diameter at the proximal end is larger than that of the distal end, better matching the calibers of the CCA and the ICA, respectively. In one embodiment, tapered stents can have a gradual, conical taper or a more abrupt, shouldered taper. Other examples may be aorto-iliac and ilio-femoral transition in the arterial side and ilio-caval transition in the venous side.

The disclosed stent may be considered as a bare-metal stent. It may partially or substantially be constructed of nitinol, and may or may not comprise a metal, metal alloy, and/or biodegradable material. For example, combining nitinol with any number of core materials may provide a nitinol composite with significantly increased properties (such as radiopacity, conductivity, or resiliency) while still maintaining its elasticity. Various materials may be paired with nitinol for portions of the disclosed stent, including but not limited to stainless steel, nickel, titanium, gold, platinum, tantalum, palladium, and alloys thereof.

In one embodiment, based on the arterial version of the nitinol woven stent (e.g., SUPERA), the stent should be modified according to the special requirement of the intended anatomical body (e.g., arterial, venous, etc.). For example, there are several ways to achieve the ideal radial force/hoop strength for the venous stents. In some instances, however, excellent expansile force is a must (e.g., under the inguinal ligament or the treatment of the May-Turner syndrome). Careful selection and combination of the diameter of the nitinol wire strands, the number of wire strands used for the structure, and angle between the crossing wires offer plenty of possibilities to find the ideal strength. Simultaneously, the stent's other characteristics can also be modified according to the needs of the special application. For example, the woven structure allows for creating a closed cell design or an open cell design and any version in between.

FIG. 2B illustrates one schematic of a stent according to one embodiment of the present disclosure. Similar to FIG. 2A, FIG. 2B illustrates stent 250 that is a self-expanding stent and comprises cylindrical body 251 with a first end and a second end. In contrast to stent 200, stent 250 comprises a second set of wires (as opposed to just one set of wires) to form the stent structure. Body 251 may have a substantially tubular shape, uniform shape, or a substantially tapered shape, among other configurations. In one embodiment, body 251 may be formed of a first plurality of wires 261 and a second plurality of wires 263 that are each woven together to form a mesh or grid body for stent 250. Angle alpha (α) illustrates the relative angle of the mesh between crossing wires. As illustrated in FIG. 2B, the second set of wires run generally parallel to the first set of wires. Regarding formation of the stent, the second set of wires are attached to the template in a similar fashion as the first set of wires. In one embodiment, a second set of pins may be located just below the first set of pins to connect the second set of wires. In general, the weave illustrated in FIG. 2B is substantially similar in shape, size, and/or overall configuration as the weave illustrated in FIG. 2A, but for simplicity some of the same components (such as closed structures 221) in FIG. 2A are not labelled in FIG. 2B.

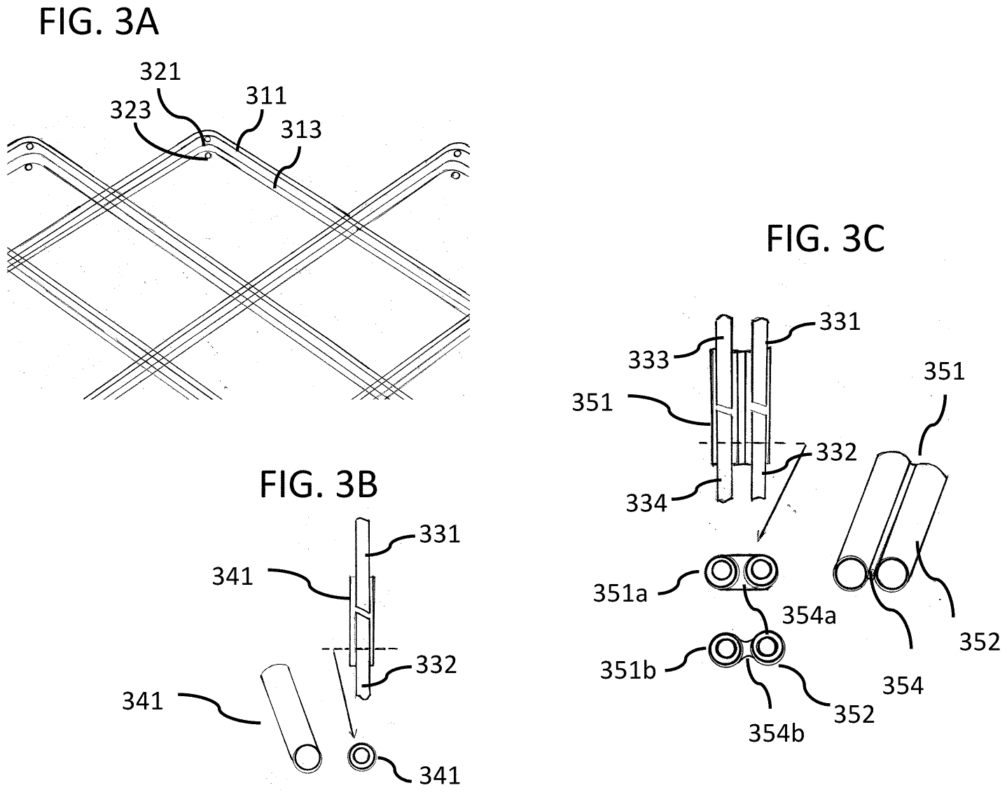

FIGS. 3A-3C provide more detail on the wires and connecting pieces of the wires from the embodiment illustrated in FIG. 2B. FIG. 3A illustrates one schematic of a wire mesh of the stent from FIG. 2B made from two parallel wires, while FIGS. 3B and 3C illustrate various schematics of a connecting piece for a stent. FIG. 3A essentially illustrates an enlarged view of a section of the weave from FIG. 2B showing two parallel wires. In one embodiment, first wire 311 is substantially parallel to second wire 313. Each of the wires is bent around a pin to form the angled weave of the stent. For example, first wire 311 bends around pin 321 and second wire 313 bends around pin 323 to form an angle. This pattern continues along the length of the stent body, thereby forming a weave pattern to the stent. Angle alpha (a) illustrates the relative angle of the mesh between crossing wires. The two-wire stent is back woven, and a connecting piece (not shown in FIG. 3A) is used to unite the respective wire ends. A connecting piece for a one-wire stent is illustrated in FIG. 3B. Connecting piece 341 is essentially a cylindrical element that connects wire portion 331 to wire portion 332. A cross section of the connecting piece illustrates that it has a substantially circular cross section. A connecting piece for a two-wire stent is illustrated in FIG. 3C. Connecting piece 351 is essentially a first cylindrical element that connects wire portion 331 to wire portion 332 and a second cylindrical element that connects wire portion 333 to wire portion 334. As seen in FIGS. 3A and 3C, the ends of the wires, which are covered by connecting piece 350, may be beveled in one embodiment. In one embodiment, connecting piece 351 comprises two cylindrical elements 352 connected by strut 354. These configurations help to ensure a fixed position for the wire ends inside cylindrical elements 352 during welding and/or crimping. The strut may vary in size, as illustrated by cross-sectional views 351a and 351b.

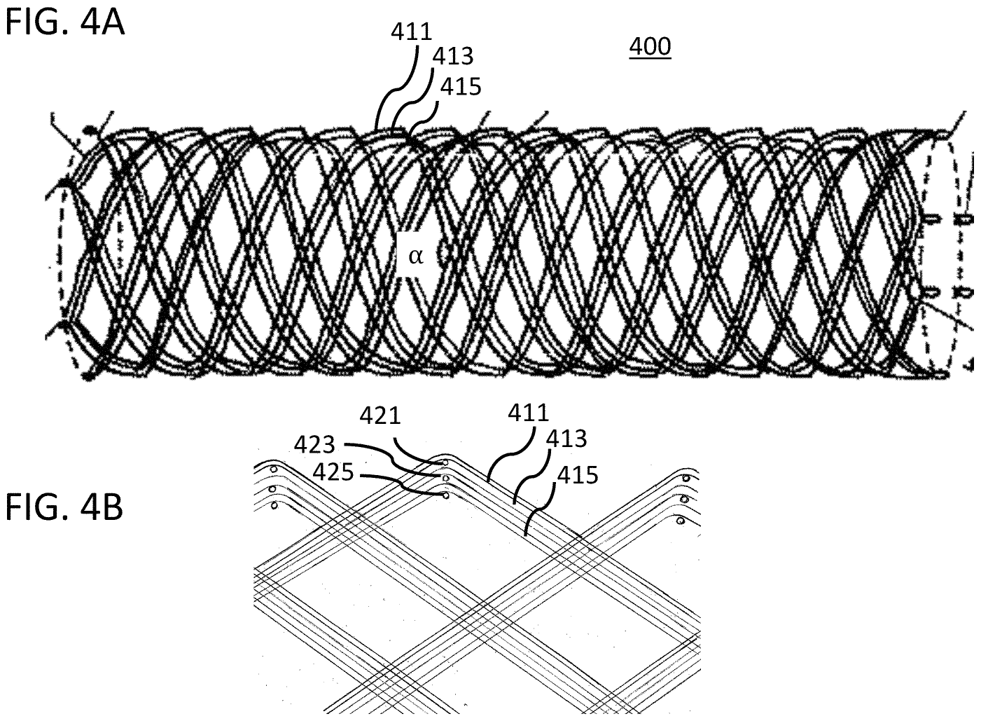

In still other embodiments, the stent may utilize more than two parallel wires. For example, FIGS. 4A and 4B illustrates a stent that utilizes 3 parallel wires. In particular, FIG. 4A illustrates one schematic of a stent according to one embodiment of the present disclosure, and FIG. 4B illustrates an enlarged version of the wire mesh from FIG. 4A. In one embodiment, stent 400 comprises first wire 411, second wire 413, and third wire 415, with each of the wires arranged substantially parallel to each of the other wires in forming the overall weave of the stent. Comparing FIGS. 2A, 2B, and 4A, the weaves remain substantially the same. Each of the wires is bent around a pin to form the angled weave of the stent. In this embodiment, a third set of pins is attached to the cylindrical template for forming the woven stent. For example, first wire 411 bends around pin 421, second wire 413 bends around pin 423, and third wire 415 bends around pin 425. This pattern continues along the length of the stent body, thereby forming a weave pattern to the stent. Angle alpha (a) illustrates the relative angle of the mesh between crossing wires. Similar to FIG. 3B, each of the respective wire ends is connected using a triple cylindrical connective piece (not shown) that comprises three cylindrical elements connected by adjacent struts.

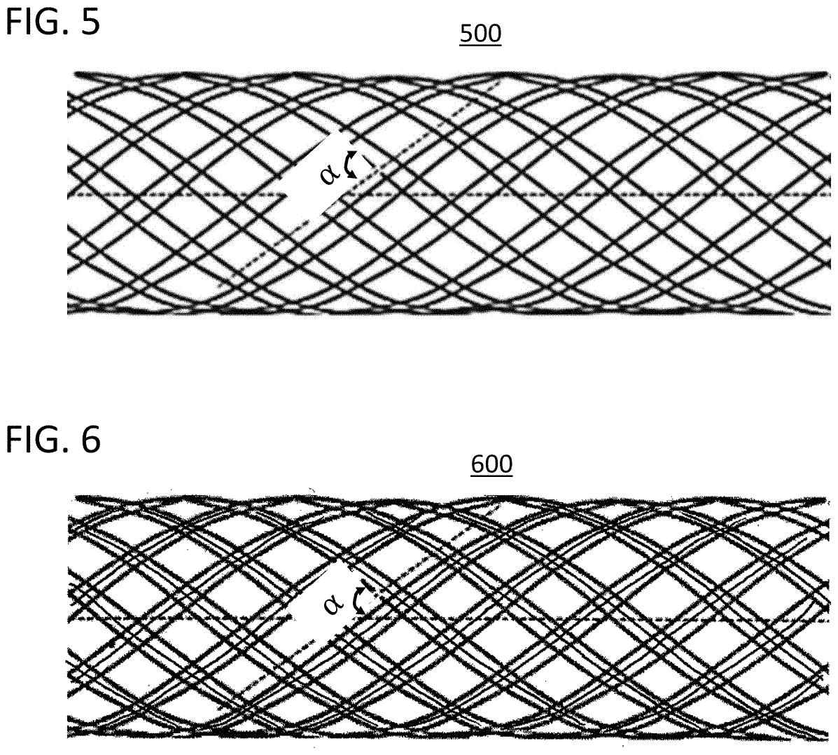

As discussed above, the weave structure of the stents may be varied. For example, the distance between adjacent parallel wires may be increased or decreased. As another example, the angles between the crossing wires may be increased or decreased. For instance, the angles illustrated in FIGS. 2A, 2B, and 4A are obtuse, thereby creating a mesh that is tight. In other embodiments, the angles between the crossing wires may be substantially acute, thereby creating a mesh that is much looser. For example, FIGS. 5 and 6 illustrate a schematic of stent 500 and stent 600, respectively, that has a mesh structure that is more acute than the mesh structures of FIGS. 2A, 2B, and 4A. As illustrated, FIG. 5 shows a two-wire parallel stent structure and FIG. 6 shows a three-wire parallel stent structure according to one embodiment of the present disclosure. Angle alpha (α) illustrates the relative angle of a cell between crossing wires within a wire mesh as measured by two adjacent sides within a rhomboid shape of the mesh along the longitudinal access of the stent.

In one embodiment, using multiple parallel wires for the disclosed stent as mentioned above allows for using smaller caliber (e.g., weaker) wires. This is beneficial for multiple reasons. For example, the weaker wires may result in a decreased expansile force of the stent. This feature can be used when a robust radial force or crush resistance is not needed (e.g., in some locations in the venous system a weaker expansile force is desired). In one embodiment, the decrease in the radial force can be partially compensated by the multiple crossings of the multiple wires. In another embodiment, the coverage of the stent and/or the mesh angle can also be adjusted according to the requirements of the given anatomy. For example, a close cell mesh (e.g., FIGS. 2A, 2B, and 4A) or an open cell mesh (e.g., FIGS. 5 and 6) can be selected. In one embodiment, a close cell mesh is one with alpha angles greater than 90 degrees, and an open cell mesh is one with alpha angles less than 90 degrees.

### Multiple Wire Pairs

In one embodiment, the disclosed stent may utilize an increased number of wire pairs and/or pins to vary the mesh size. In one embodiment, additional pins may be arranged circumferentially or vertically on a cylindrical template for formation of the stent. In one embodiment, the additional pins are positioned on the same level of the template around a circle. Consequently, the total number of wires that form the mesh can be increased. For example, instead of using 6 wire pairs (12 wires), one can use 8 pairs (16 wires), 10 pairs (20 wires), or even 24 pairs (48 wires), as well as any other reasonable number of pairs/wires to form the mesh. Another possibility is that the additional pins are positioned below the original pins to form a second circle. This variation will result is a slightly decreased radial force at the end of the stent where the mesh is formed on only the first (outer) circle of pins. Thus, the mesh tightness will be much looser here. The number of wires in this latter arrangement may vary in a wide range. All these modifications serve the same goal to create the most optimal physical characteristics of the stent for the given anatomical structure.

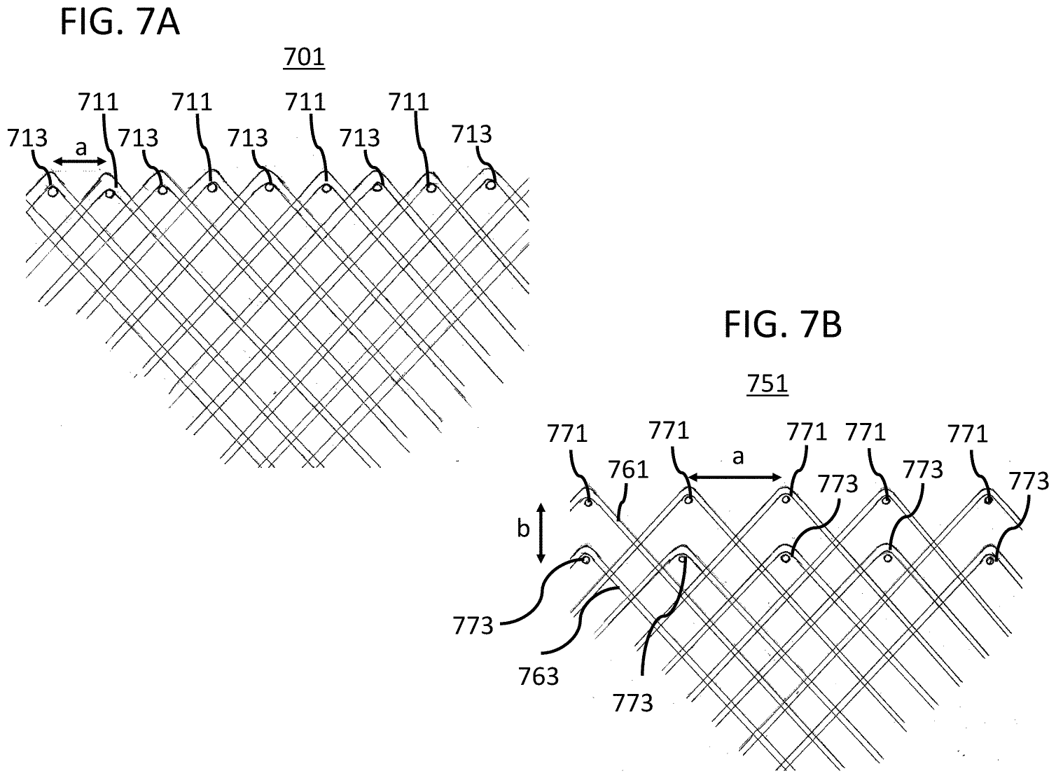

FIG. 7A illustrates one schematic of a wire mesh pattern of a stent according to one embodiment of the present disclosure, whereby the number of wire pairs is increased by attaching more pins on the circumference of a cylindrical template. In one embodiment, rather than using six pins for six wire pairs (e.g., 12 wire strands), an increased number of pins for the same number of wire pairs may be utilized and attached to the mandrel, such as eight, ten, twelve, or twenty-four pins. The resulting weave creates a tighter mesh and larger coverage of the vessel. As an example, FIG. 7A shows wire weave 701 with wire bending over a plurality of pins. First set of pins 711 may be illustrative of the standard spacing on conventional weaves for the pins. In the disclosed embodiment, additional pins 713 may be added along the circumference thereby decreasing the distance “a” between each pin. Inversely, the number of pins and the associated wire pairs can be reduced, depending on the desired weave configuration and stent properties. For example, rather than using six wire pairs, three, four, or five wire pairs can produce a strong stent depending on the diameter of the wire and the mesh tightness created. As an example, a coronary stent can be created with only three wire pairs (six wires) woven with angles of greater than approximately 140 degrees between the crossing wires, which will result in a closed cell mesh design with an adequate radial/expansile force.

FIG. 7B illustrates one schematic of a wire mesh pattern of a stent according to one embodiment of the present disclosure, whereby the number of wire pairs is increased by attaching more pins vertically on a cylindrical template. Rather than adding additional pins circumferentially as shown in FIG. 7A, the additional set of pins are located below (that is, located proximally to) the first set of pins. As an example, FIG. 7B shows wire weave 751 with first wire 761 bending over a first plurality of pins 771. First set of pins 771 may be illustrative of the standard spacing on conventional weaves for the pins arranged circumferentially. In FIG. 7B, additional pins 773 may be added proximal to the first set of pins 771. Second wire 763 may bend over the second plurality of pins 773. A proximal distance “b” may exist between each set of pins, while a longitudinal distance “a” may also exist between each set of pins. In other embodiments, the second set of pins may vary both in a distance “a” and/or “b” from the first set of pins. The resulting weave may be less tight than a conventional weave, result in less overlapping between the wires, and less area coverage. In one embodiment, this type of stent modification can be used for creating a flanged portion of the stent from this end of the stent. When a flange is created from the proximate portion of the stent, the loosely structured outer portion (defined by the wire bends attached to the first set of pins), will protrude outward more than the other part of the flange. These protrusions (wire loops) will increase the covered area of the flange and therefore increase the contact surface around of the orifice (ostium) of the side branch. Such a stent with a flange can be utilized as an ostial stent that can treat lesions that are located in the initial portion of a branch. A straight stent cannot cover the orifice, that is, the portions of the main vessel adjacent to the orifice of the side branch. In this location, the atherosclerotic plaque(s) of the main vessel shows contiguity with the lesions located in the initial portion of the side branch. In the lack of the flange, some parts of the lesion will remain untreated and/or uncovered by the straight stent. Further, the flange stent can also be an indispensable part of a modular stent assembly, where the flange stent is deployed through the mesh of an already deployed stent within the main (parent) vessel. In this manner, complex lesions (e.g., branching, bifurcations) can be efficiently treating and any complex anatomy can be remodeled. A stent with a looser mesh end can also be used in the close proximity of a side branch. The looser mesh will cause less jailing (e.g., obstructing the flow into the side branch).

### Wire Bundles

In one embodiment, the disclosed stent may utilize a bundle of wires (e.g., a plurality of individual wires coupled together) instead of a single wire. The bundle of wires may comprise different sized wires and/or wires of different materials. In one embodiment, rather than using a single filament or wire strand, a wire rope and/or wire bundle may be formed of a plurality of individual wire strands that are coupled together.

A bundle of wires instead of a single wire provides numerous benefits. For example, if one of the plurality of wires fails, the other wires can easily take up the load. Thus, any flaws in an individual wire is not as critical as compared to looking at the bundle of wires as a whole. A bundle of wires also prevents fatigue of the single individual wire, similar to the principle of rope wires (which use braided strands of individual rope filaments). This embodiment is valid for woven stent designs and not for prior art slotted tube stents. This type of stent is particularly useful for stents used in femoropopliteal arteries as well as generally venous stents, which require a longer life expectancy of the stents and increased repetitive forces leading to fatigue. In the case of venous applications, the stent's life-expectancy and fatigue-freeness is a critical issue because venous diseases require stent placements at a much younger age in comparison with arterial applications (arteriosclerosis). Therefore, the venous stents should serve without failure for 40-50 years compared to the 5-20 years for the arterial stents. The prior art arterial stents and even the recently developed specific dedicated venous stents have not proven themselves in that regard. The potential for failure in encoded into the structure of these stent: the slotted tube designs are inherently inferior because their flexibility is achieved by creating connecting elements (bridges) that in turn make the stents extremely vulnerable and prone to fracture in the long run.

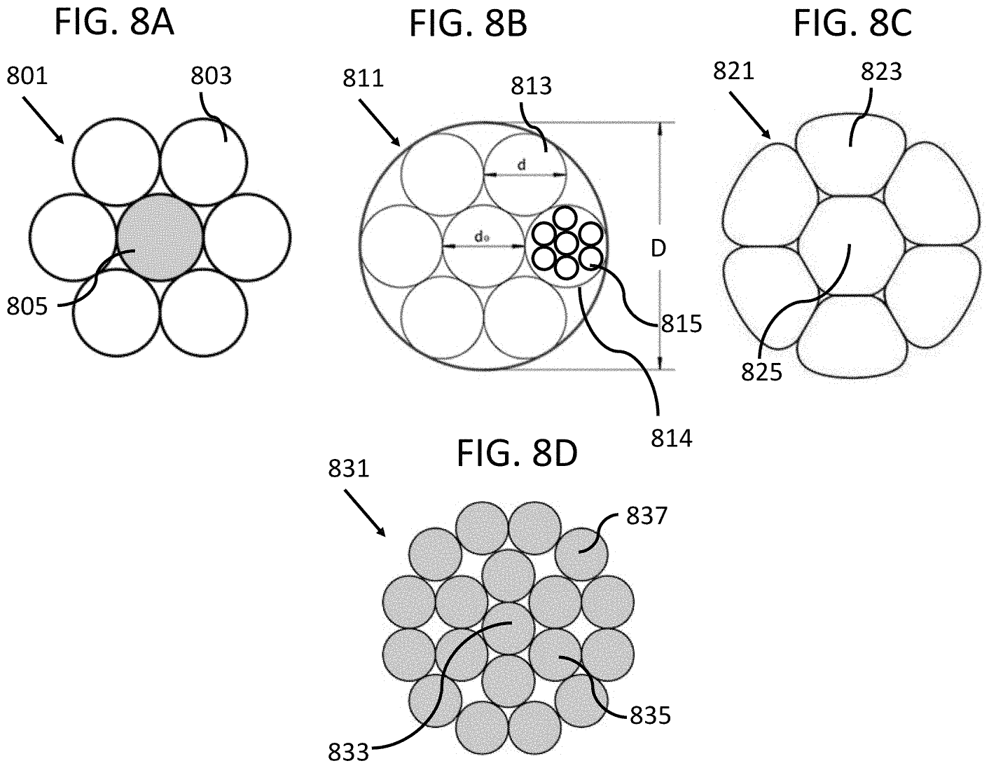

FIGS. 8A-8D illustrate various cross-sectional schematics of wire bundles according to one embodiment of the present disclosure. Each of these wire bundles may be used to form a wire of the woven stent as illustrated previously.

FIG. 8A illustrates wire bundle 801, which may be formed of seven individual wire strands 803. Each of these strands may be twisted together helically. In one embodiment, each of the wires in the wire bundle touches a plurality of adjacent wires. In one embodiment, the central wire 805 is surrounded by approximately six additional wires. In one embodiment, one or more of the wires may comprise a nitinol microtube that may contain a platinum core used as a radiopaque marker, which is used to increase the visibility of the stent on fluoroscopy (X-ray) during deployment. For example, central wire 805 may comprise a microtube with a platinum core. In other embodiments, all of the wires may contain microtubings with platinum cores.

FIG. 8B illustrates the wire bundle of FIG. 8A with exemplary dimensions. Each of the seven individual wires 813 may have approximately 0.003″ diameter (d), resulting in an overall diameter (D) of wire bundle 811 of approximately 0.009″. In another embodiment, one or more of individual wires 811 may comprise a plurality of smaller individual wires 815, thereby creating a more complex but stronger wire bundle (again, much like a braided rope). In one embodiment, one or more of wires 813 may comprise at least three wires 815, which each may comprise a diameter of 0.001″ thereby keeping the overall diameter of wire 813 at approximately 0.003″. In one embodiment, inner wire bundle 814 comprises approximately seven smaller diameter wires 815. Of course, other diameters and sizes of wires may be utilized as would be known to one of ordinary skill in the art based on the present disclosure.

FIG. 8C illustrates another schematic of a wire bundle according to one embodiment of the present disclosure. Wire bundle 821 may be substantially similar to wire bundle 801 but comprises one or more wires that are flattened, non-symmetrical, and/or non-cylindrical. For example, a substantially cylindrical wire 825 may be surrounded by a plurality of flattened wires 823 that are not cylindrical, thereby providing an overall compacted shape of wire bundle 821. Because these wires are flattened and/or non-symmetrical, there is not a constant diameter of each wire as compared to FIG. 8B.

FIG. 8D illustrates another schematic of a wire bundle according to one embodiment of the present disclosure. Wire bundle 831 may be substantially similar to wire bundle 801 but comprises additional wires. For example, wire bundle 831 may comprise center wire 833, a second set of wires 835 (such as six wires) surrounding the center wire, and a third set of wires 837 (such as 12 wires) surrounding the second set of wires 835. In the embodiment illustrated in FIG. 8D, there are approximately 19 individual wires, each of which may be comprised of individual wires itself. This embodiment is similar to wire bundle 801 but adds an outer peripheral set of wires 837. In this embodiment, there is an outer set of wires and an inner set of wires that together form wire bundle 831.

There are numerous design variations of a wire bundle that can be used in a woven stent, depending on the intended stent configurations, application of the stent, and anatomical location in which the stent is to be deployed. For example, some of the wires may be different shapes and/or sizes, while some of the wires may be different materials. Following the principle of the wire rope, virtually endless variations of a wire bundle can be created that can be used as individual wire strands for a woven stent.

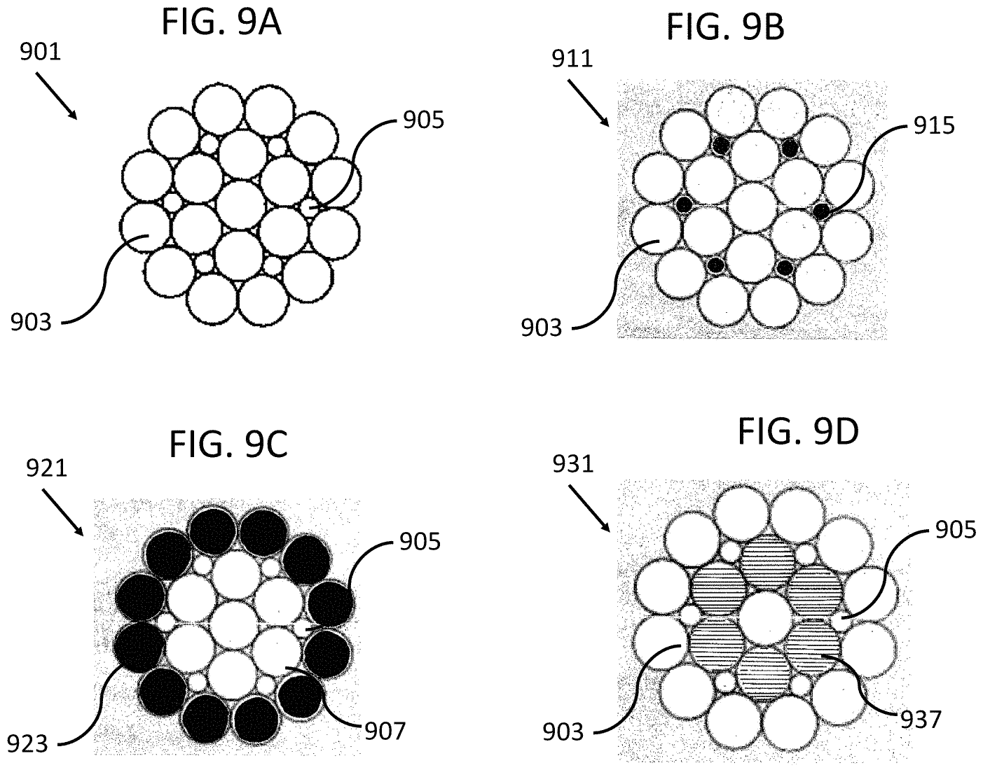

FIGS. 9A-9D illustrate various schematics of wire bundles according to one embodiment of the present disclosure. These embodiments are similar to the embodiments illustrated in FIGS. 8A-8D but have varying sizes of wires in the wire bundle.

FIG. 9A illustrates a wire bundle with a plurality of different sized wires according to one embodiment of the present disclosure. In one embodiment, wire bundle 901 may be substantially similar to wire bundle 831 but adds a plurality of smaller caliber/diameter wires to the wire bundle. For example, wire bundle 901 may comprise a plurality of wires 903 in a plurality of concentric radial positions, and between some of the wires a plurality of smaller diameter wires 905 may be located to add an intended benefit/capability to the overall wire bundle. The wire bundle illustrated in FIG. 9A comprises approximately 25 wires, with approximately 19 wires being the same sized wires as illustrated in FIG. 8D and six of the wires being smaller diameter wires located between and/or adjacent some of the larger diameter wires. In one embodiment, each smaller diameter wire is surrounded by approximately four larger diameter wires. In one embodiment, each of the larger diameter wires may itself be formed of a plurality of smaller diameter wires. In one embodiment, the composition of the smaller diameter wires 905 is different than a composition of the larger diameter wires 903. In one embodiment, each of the wires in the wire bundle touches a plurality of adjacent wires.

FIGS. 9B-9D illustrate various embodiments of the wire bundle from FIG. 9A with radiopaque markers according to one embodiment of the present disclosure. FIG. 9B illustrates wire bundle 911 with smaller diameter wires 915 comprising platinum cored microtubing. FIG. 9C illustrates wire bundle 921 with a portion of the larger diameter wires 903 comprising platinum cored microtubing. In this embodiment, outer circle wires 923 are the radiopaque markers while the inner circle wires 907 have no enhanced radiopacity. Inversely, FIG. 9D illustrates wire bundle 931 with inner circle wires 937 being radiopaque markers comprising platinum cored microtubing while the outer circle wires 903 and smaller diameter wires 905 have no enhanced radiopacity. In another embodiment (not shown), a central wire may be intensively radiopaque, similar to the wire bundle shown in FIG. 8A. In each of the embodiments of FIGS. 9A-9D, there is an outer set of wires and an inner set of wires that together form the relevant wire bundle.

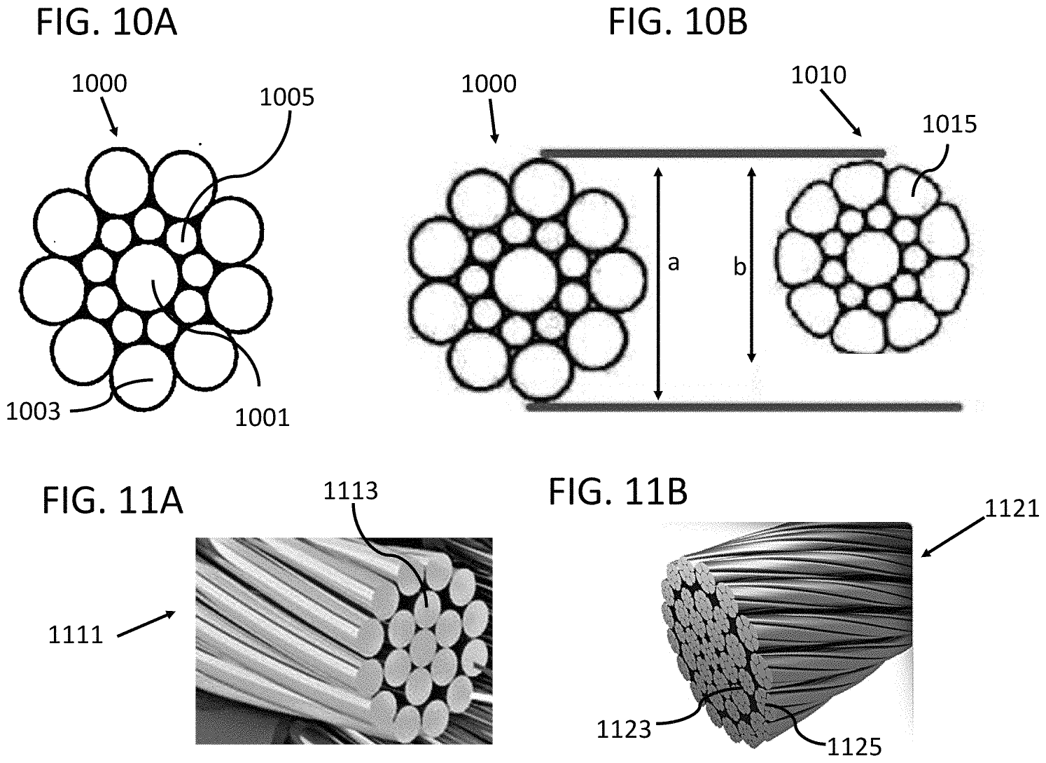

FIG. 10A illustrates a wire bundle with a plurality of different sized wires according to one embodiment of the present disclosure. In this embodiment, wire bundle 1000 comprises at least two different sized wires. In one embodiment, center wire 1001 is a larger diameter wire surrounded by a plurality of smaller diameter wires 1005, which is surrounded by an outer set of wires 1003 with a relatively large diameter. In one embodiment, the diameter of inner wire 1001 is the same as the diameter of outer wires 1003. In one embodiment, center wire 1001 is surrounded by approximately eight smaller diameter wires 1005, which are surrounded by approximately nine larger diameter wires 1003.

As is known in the art, the diameter of the wire used for a woven structure determines the size of the delivery system. The delivery system of a stent consists of an inner and an outer catheter. In one embodiment, the outer diameter (OD) of the delivery system can be described by using the following equation: OD total=OD inner catheter+4× wire diameter (assuming two crossing wires are on both sides of the inner catheter)+wall thickness of the outer catheter. Thus, reduction of the wire diameter greatly decreases the needed diameter of the delivery system. However, in general, conventional wires cannot be decreased significantly without materially impairing the strength of the wire strand and the overall stent. In one embodiment, utilizing wire bundles instead of individual wire strands allows the overall wire thickness to decrease, which among other benefits, reduces the overall diameter of the delivery system needed.

Another embodiment on reducing the diameter of the wire bundle is to make the wire bundle/rope in a compacted fashion. FIG. 8C illustrates one such compacted wire, in which some of the individual wires are shaped to minimize the dead space within a wire bundle which exists when each of the wires are cylindrical/symmetrical. Similarly, FIG. 10B illustrates a compacted wire bundle of the wire bundle schematic illustrated in FIG. 10A. In one embodiment, compacted wire bundle 1010 is substantially similar to wire bundle 1000, but outer wires 1015 are compacted and/or symmetrical and/or non-circular as compared to outer circle wires 1003. In other words, wires 1015 do not have a uniform diameter. In one embodiment, using non-symmetrical outer wires 1015 allows the overall diameter of wire bundle 1011 to be reduced from diameter “a” (for bundle 1000) to diameter “b,” while the overall amount of wire material and strength remains substantially the same between the different bundles.

In one embodiment, the wire bundle may be a twisted and/or helical wire bundle. FIGS. 11A and 11B illustrate various schematics of a twisted wire bundle according to one embodiment of the present disclosure. FIG. 11A illustrates wire bundle 1111 that is substantially similar to the wire bundle of FIG. 8D, but each of wires 1113 is twisted to form an overall twisted and/or helical wire bundle. FIG. 11B likewise illustrates a twisted wire bundle 1121 according to another embodiment. However, in this embodiment each of the individual wires 1123 that form wire bundle 1121 comprises its own wire bundle and a set of individual wires 1125. Thus, wire 1123 can be considered a first wire bundle with a smaller diameter than wire bundle 1121, and wire 1125 can be considered to have a smaller diameter than wire bundle 1123. While wire bundle 1121 may be twisted (based on twisting wire bundles 1123), the individual wires 1125 within wire bundle 1123 may or may not be twisted.

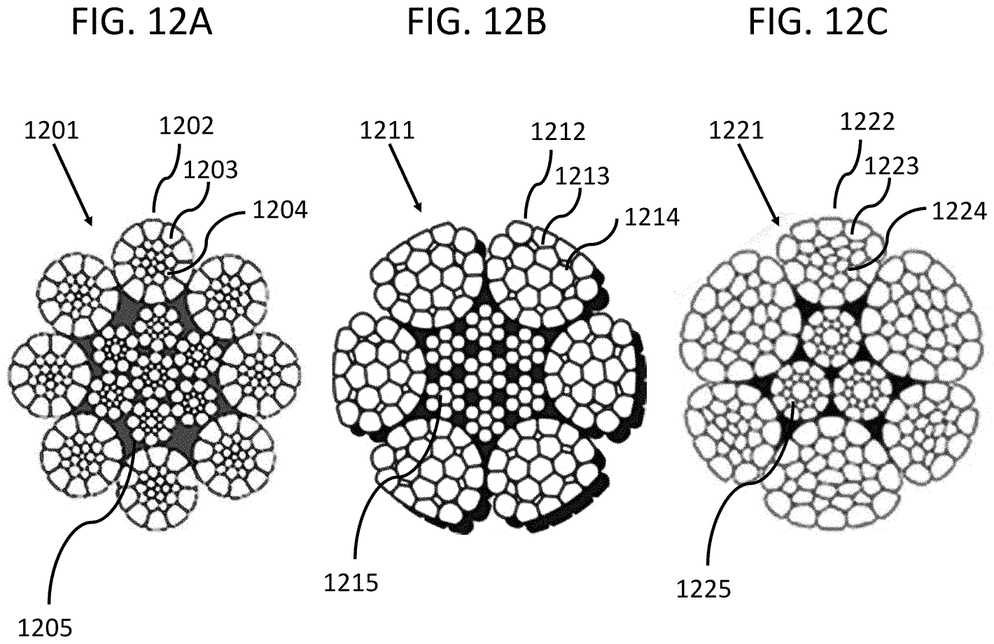

In one embodiment, the wire bundle may comprise an inner set of wire bundles and an outer set of wire bundles in various complex shapes and/or configurations. FIGS. 12A-12C illustrate various schematics of wire bundles according to one embodiment of the present disclosure.

FIG. 12A illustrates complex wire bundle 1201 that comprises an outer set of substantially round wire bundles 1202 (such as eight outer wire bundles) and an inner core of wire bundles 1205 (such as seven inner wire bundles). Outer wire bundles 1202 comprise an outer set of partially compacted wires 1203 and an inner set of round wires 1204. Inner wire bundles 1205 may be constructed from an outer set of wires and an inner set of wires, wherein the outer and inner sets of wires may be different diameters and/or materials. In one embodiment, the inner wire bundles may have a central wire.

FIG. 12B illustrates complex wire bundle 1211 that comprises an outer set of substantially compacted wire bundles 1212 (such as six outer wire bundles) and an inner core of wire bundles 1215 (such as seven inner wire bundles). The outer wire bundles may comprise substantially compacted wires 1213 of similar diameters, and may or may not include smaller diameter wires 1214. Inner wire bundles 1215 may be constructed from wires (e.g., seven) of approximately the same diameter.

FIG. 12C illustrates complex wire bundle 1221 that comprises an outer set of substantially compacted wire bundles 1222 (such as six outer wire bundles) and an inner core of wire bundles 1225 (such as three inner wire bundles). Outer wire bundles 1222 may comprise substantially compacted wires 1223, 1244 of different diameters. In one embodiment, outer wire bundles 1222 may comprise a first set of bundles of a first wire size and a second set of bundles with a second wire size, where the first and second wire sizes are different. As illustrated, the differently sized outer wire bundles may be arranged alternatively, such that a smaller wire bundle is adjacent to two larger wire bundles, and vice versa. Inner wire bundles 1225 may be constructed from three round wire bundles, each which comprises a plurality of different sized and shaped wires. For example, each of the inner wire bundles may have substantially compacted outer wires and smaller substantially round wires forming an inner circle around a central wire. As seen, the variations of wire bundles for a wire that can be utilized in the disclosed stent is large, and depends on the particular desired strengths and features of the wire, wire bundle, and overall stent.

### Bioresorbable Stent

In one embodiment, the disclosed stent may be a bioresorbable stent. In general, a bioresorbable stent has the capability to degrade within six to twenty-four months within the body. They can be used as a temporary scaffold and no material is left behind after they degrade (in contrast to metal stents). In general, bioresorbable vascular scaffold (BVS) technology has been used in the coronary arterial system. Scaffolds have been either polymer-based or metallic-based. The most common polymer-based scaffold is made from poly-L lactic acid, the material used in absorbable sutures and biologic implants. Bioresorbable scaffolds provide vessel support and potential antiproliferative drug delivery. Theoretically, their ability to absorb over time improves vessel conformability and potentially may restore the vessel to its physiologic state. Bioresorbable scaffolds offer less concern of jailing side branches, and there would be a decrease risk of late stent fracture and late stent thrombosis. Also, the scaffold would have less magnetic resonance and computed tomography imaging artifact.

The Absorb BVS (offered by Abbott Vascular) is one type of bioresorbable stent/scaffold, but studies have shown various adverse effects include thrombosis. Technical concerns of Absorb BVS deployment included difficult delivery of the system due to thickness and maneuverability in small tortuous arterial vessels, difficult visualization despite presence of platinum radiopaque markers, need for adequate predilation to allow for full scaffold expansion, recoil after expansion, and inability to over-dilate due to scaffold damage.

In one embodiment, the disclosed wire bundles (e.g., coupled/stranded wire filaments instead of a single wire) can be used to make various stents (such as the Absorb BVS stent) more resistant to overdilation based on the wire bundle principles disclosed herein. Using the wire bundles, the tensile strength of the overall wire bundle is significantly increased as compared to a similar sized individual wire. The wire/filament bundle is stronger than the individual strut and if it is made in a compacted fashion (see, e.g., FIGS. 8C and 10B) the bulkiness of the stent can also be decreased. Still further, the flexibility of such a stent can also be improved by using the disclosed wire/filament bundles.

### Wire Mesh

In one embodiment, the angles in the wire mesh of the disclosed stent can be varied on a wide scale from a very compact mesh tightness (such as where the axially oriented angles approaching 180 degrees) to a very open cell design (which may have axially oriented angles less than 90 degrees). For example, the angles illustrated in FIGS. 5 and 6 show an open cell design. These changes in the mesh tightness are based partly on the variations of the disclosed self expanding stents that are created with additional wires and pins and/or with multiple parallel wires and/or bundled wires.

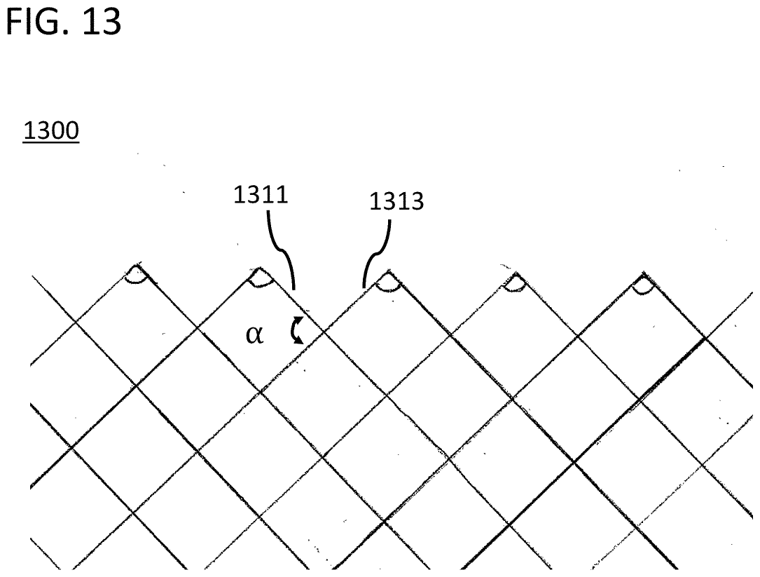

In one embodiment, the disclosed stent may utilize crossing wires that form an angle of approximately 90 degrees. In contrast, the conventional Supera stent has a woven structure that has obtuse angles. See, e.g., the '401 Patent. For example, FIG. 13 illustrates mesh 1300 for a woven stent with wires 1311, 1313 forming an angle of approximately 90 degrees where they cross. This stent can be formed of a single wire or multiple wires, or even bundled wires as described herein. For example, a stent may be formed with multiple parallel wires with 90 degrees between the respective wires, similar to the two wire and three wire meshes illustrated in FIGS. 3A and 4B.

In one embodiment, the angles of the stent mesh provide various groups based on the angle sizes. For example, a first stent group (the “acute angle group”) may have axially oriented angles that are less than 90 degrees. In one embodiment, the angles may be at least 60 degrees, and may be greater than 60 degrees and less than 90 degrees. In some embodiments, the angle may be less than 60 degrees. A second stent group (the “obtuse angle group”) may have axially oriented angles that are greater than 90 degrees. In one embodiment, the angles may be at least 90 degrees and less than 180 degrees. A third stent group may have axially oriented angles that are approximately 90 degrees.

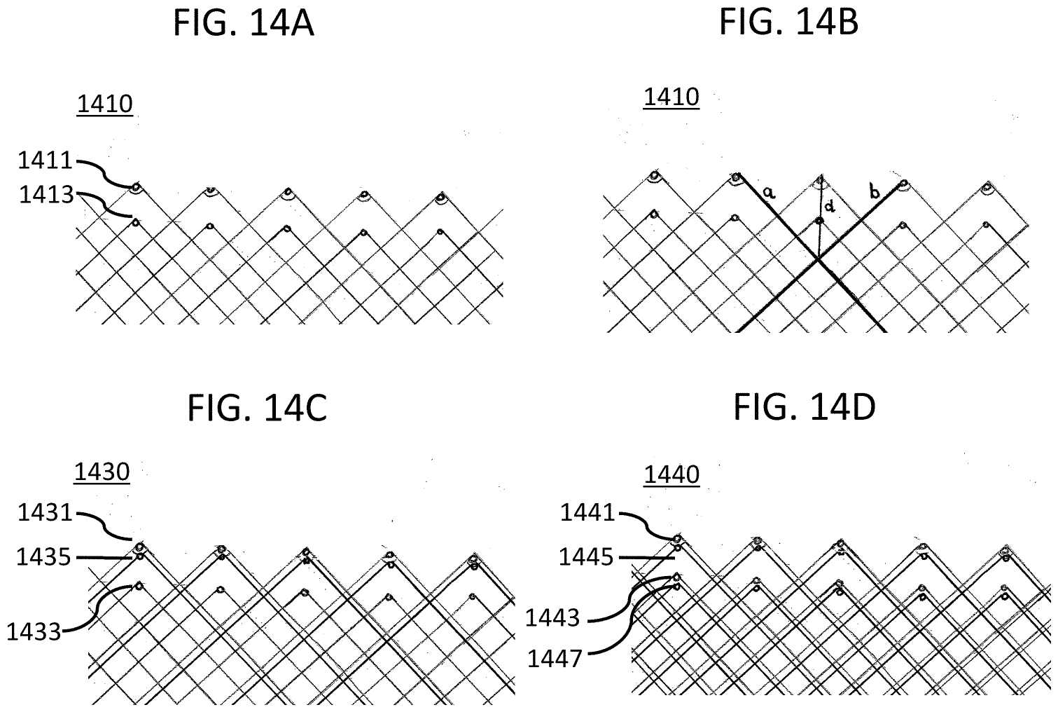

FIGS. 14A-14D illustrate various schematics of wire meshes according to one embodiment of the present disclosure.

FIG. 14A illustrates wire mesh 1410 with an angle alpha of approximately 90 degrees between the crossing wires. In this embodiment two sets of pins utilized. First set of pins 1411 is arranged along the circumference of the template adjacent to its very end. Second set of pins 1413 are arranged vertically in a way below the first set of pins 1411. In this embodiment, the second set of pins is substantially beneath the first set of pins. FIG. 14B is the same wire mesh as found in FIG. 14A. As illustrated in FIG. 14B, the second set of pins is located at the points that are approximately halfway in the distance “d” between the wire crossings formed by the wires “a” and “b” that are bent on the immediate neighboring pins located in the first set of pins.

FIG. 14C illustrates another embodiment of wire mesh 1430 with angles of approximately 90 degrees between the crossing wires. The wire mesh is based on three sets of pins, a first set of pins 1431, second set of pins 1433, and third set of pins 1435. In this embodiment, first and second set of pins 1431, 1433 is substantially similar to the embodiment illustrated in FIG. 14A, but adds a third set of pins 1435 close below first set of pins 1431. Additional set of pins 1435 is used as a bending point for a second wire parallel to “a” and “b” wires (see FIG. 14B). In other words, the additional set of pins 1435 allows a first set of parallel wires. Again, this embodiment differs as compared to that illustrated in FIG. 14A because this embodiment adds a second parallel wire substantially close/adjacent to the first set of wire. In this embodiment, the wires belonging to the second set of pins is a single wire.

FIG. 14D illustrates yet another embodiment of wire mesh 1440, which adds an additional set of pins 1447 vertically just below a second set of pins 1443. As illustrated in FIG. 14D, these pins are used to form a second set of parallel wires. Wire mesh 1440 is based on four sets of pins, a first set of pins 1441, second set of pins 1443, third set of pins 1445, and fourth set of pins 1447. The first row of pins 1441 and second row of pins 1443 is substantially similar to the set of pins in FIG. 14A. Third row of pins 1445 is similar to third row of pins 1435 in FIG. 14C and forms a first set of parallel wires. Fourth row of pins 1447 is located close and lower to second row of pins 1443 and forms a second set of parallel wires. Thus, wire mesh 1440 comprises two sets of parallel wires. In summary, FIGS. 14A and 14B show two sets of single wires, FIG. 14C shows one set of parallel wires and one set of a single wire, and FIG. 14D shows two sets of parallel wires. In one embodiment, all of the wires in these illustrated mesh patterns cross each other at an approximately 90 degree angle.

These embodiments can be reproduced by using substantially cored/solid wires and/or bundled wires. In one embodiment, all of the wires are bundled, while in other embodiments one or more of the wires may be bundled. Other variations exist, and the same stent structure may comprise differently structured wire bundles (both in shape, material, size, configuration, etc.). In one embodiment, each of the embodiments illustrated in FIGS. 14A-14D can be created with angles different than 90 degrees, such as acute angle stents and obtuse angle stents. Further, these embodiments can be produced in a tapered shape and/or with a flanged portion. Still further, these embodiments can be created with segments having varying mesh tightness, that is the altering closed and open cell segments in the same stent structure.

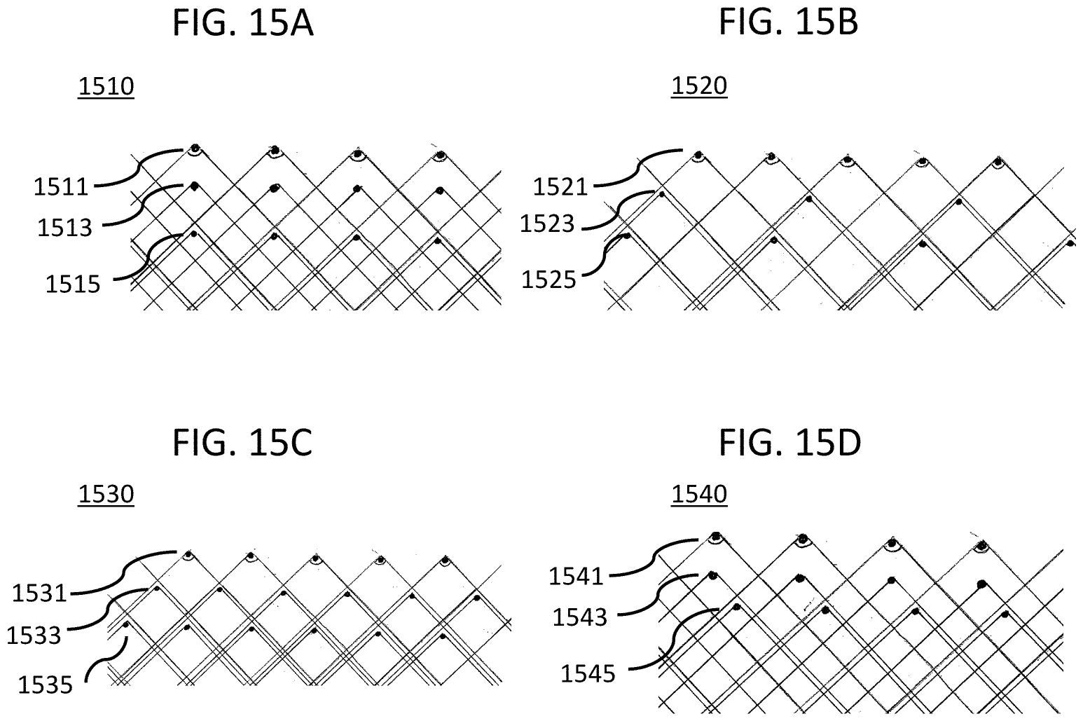

FIGS. 15A-15D illustrate various schematics of wire meshes according to one embodiment of the present disclosure, wherein the mesh uses three rows of wires. In one embodiment, all of the wires in these illustrated mesh patterns cross each other at an approximately 90 degree angle, although obtuse or acute angled mesh patterns may also be utilized with these embodiments.

FIG. 15A illustrates wire mesh 1510 wherein three rows of pins are vertically arranged. The wire mesh is based on three sets of pins, first set of pins 1511, second set of pins 1513, and third set of pins 1515. A pin on each row is substantially in-line with corresponding pins on the other rows.

FIGS. 15B and 15C illustrate a mesh wherein three rows of pins are arranged on a slant line. As illustrated in FIG. 15B, in one embodiment, wire mesh 1520 comprises a first row of pins 1521 at each position, and the second and third rows have pins in every other position (defined by the angles formed by first row of pins 1521) relative to the first row of pins. For example, second row of pins 1523 may be positioned in between a position of first row of pins 1521, and third row of pins 1525 may be positioned between a position of the second row of pins 1523 or substantially in-line with a position of the first row of pins 1521 in alternating spaces. As illustrated in FIG. 15C, wire mesh 1530 comprises first set of pins 1531, second set of pins 1533, and third set of pins 1535, and the second and third rows have pins in every possible position relative to the angles formed by first row of pins 1531.

FIG. 15D illustrates wire mesh 1540 wherein three rows of pins 1541, 1543, 1545 are vertically arranged. In one embodiment, second row of pins 1543 is in a vertical position substantially in-line with first row of pins 1541, while third row of pins 1545 are in a second “off” position in relation to the first row of pins 1541. The “off” position is a position that is not substantially in-line with one or more of the preceding rows and is not substantially in the middle of the rows (in other words, its slightly “off” of alignment with a prior row). In one embodiment, the number of pins in each of the three rows is the same.

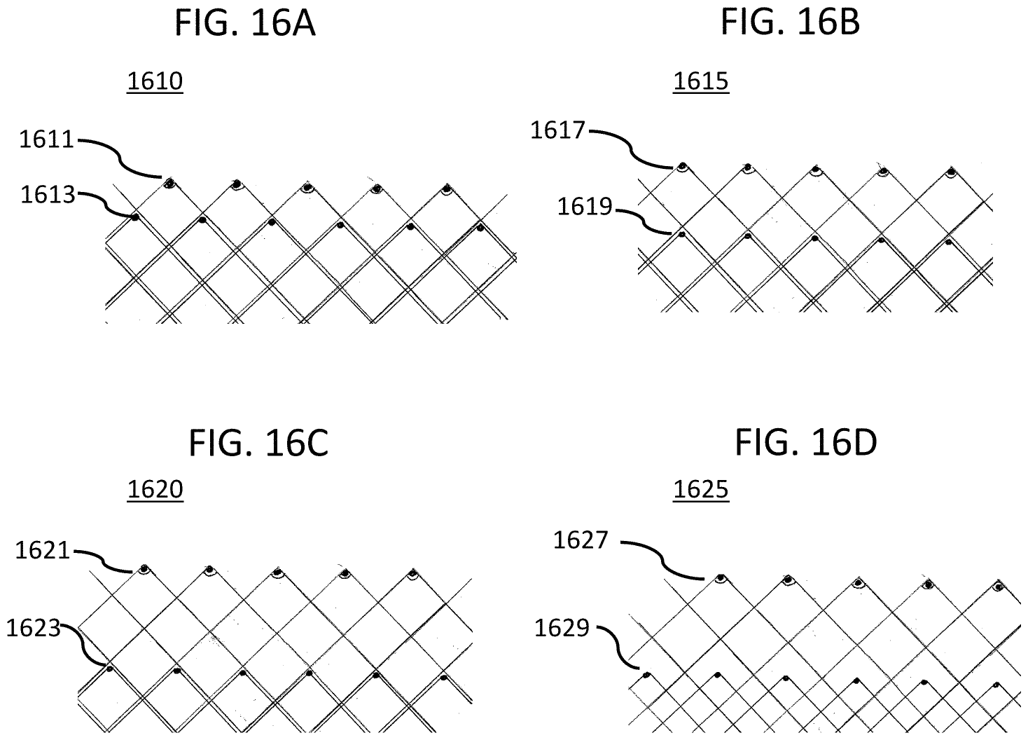

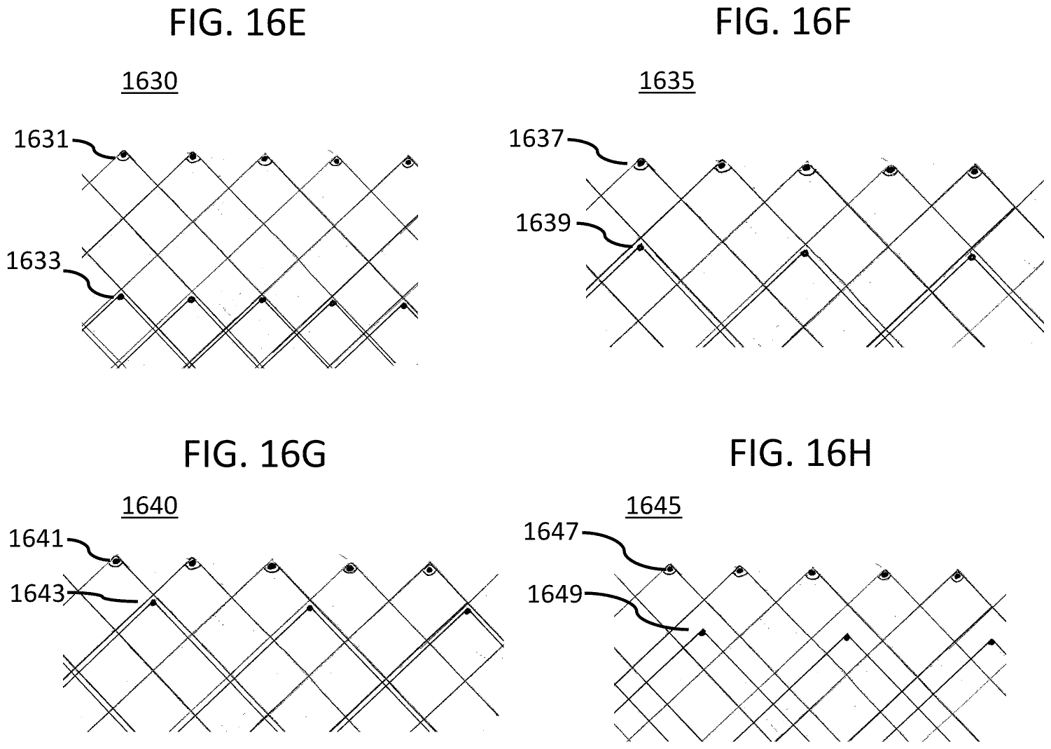

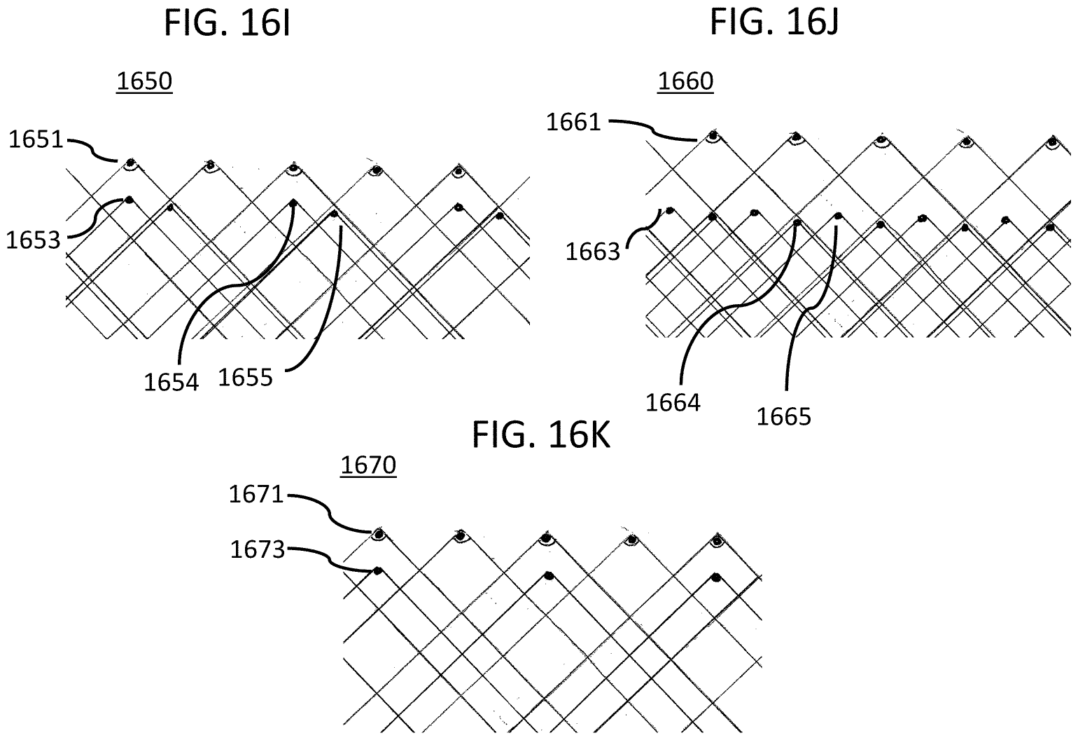

FIGS. 16A-16K illustrate various schematics of wire meshes according to one embodiment of the present disclosure, wherein the mesh uses two rows of wires. In one embodiment, all of the wires in these illustrated mesh patterns cross each other at an approximately 90-degree angle, although obtuse or acute angled mesh patterns may also be utilized with these embodiments.

FIG. 16A illustrates wire mesh 1610 with two rows of pins, which comprises first set of pins 1611 and second set of pins 1613. The second row of pins 1613 are arranged in a first “off” position relative to the first row of pins 1611. An “off” position is that which is not aligned with an earlier set of pins. In one embodiment, the second row of pins 1613 is located approximately in the middle of the first row of pins 1611.

FIG. 16B illustrates wire mesh 1615 with two rows of pins, which comprises first set of pins 1617 and second set of pins 1619. Second row pins 1619 are aligned in a vertical position relative to the first row of pins 1617.

FIG. 16C illustrates wire mesh 1620 with two rows of pins, which comprises first set of pins 1621 and second set of pins 1623. Pins 1623 on the second row are arranged in a second “off” position as compared to the first row of pins 1621. In one embodiment, the second row pins are located approximately in the middle of the pins from the first row. In comparison to FIG. 16A, there is more space between the first and second rows; in other words, there are multiple wire crossings between the first row of pins and the second row of pins. In particular, the second row of pins in FIG. 16A is located in the “first” off position wire crossings and the second row of pins in FIG. 16B is located in the “second” off position wire crossings.

FIG. 16D illustrates wire mesh 1625 with two rows of pins, which comprises first set of pins 1627 and second set of pins 1629. Pins 1629 on the second row are aligned in a vertical position relative to first row of pins 1627, but they are positioned further away from the first row of pins as compared to that illustrated in FIG. 16B, and in particular are positioned further away from a row of angles formed by the first row of pins. Further, the second row of pins 1629 are not positioned at a wire crossing for wires passing over the first row of pins 1627.

FIG. 16E illustrates wire mesh 1630 with two rows of pins, which comprises first set of pins 1631 and second set of pins 1633. The pins on the second row are aligned in a vertical position relative to the first row of pins, but they are positioned further away from the first row of pins as compared to that illustrated in FIG. 16D. Further, as compared to FIG. 16D, the second row of pins 1633 is located at wire crossings of the wire passing over the first row of pins 1631.

FIG. 16F illustrates wires mesh 1635 with two rows of pins, which comprises first set of pins 1637 and second set of pins 1639. Pins 1639 on the second row are aligned in a vertical position relative to first row of pins 1637, but the number of pins in the second row is approximately half as the number of pins in the first row. In other words, second row of pins 1639 has an empty space between each pin that corresponds to a pin 1637 on the first row.

FIG. 16G illustrates wire mesh 1640 with two rows of pins, which comprises first set of pins 1641 and second set of pins 1643. Similar to FIG. 16A, the pins on the second row are arranged in a first “off” position as compared to the pins on the first row, but the number of pins in the second row is approximately half as the number of pins in the first row. In other words, second row of pins 1643 has an empty space between each pin that corresponds to a pin 1641 on the first row.

FIG. 16H illustrates wire mesh 1645 with two rows of pins, which comprises first set of pins 1647 and second set of pins 1649. Similar to FIG. 16C, the pins on the second row are arranged in a second “off” position, but the number of pins in the second row is approximately half as the number of pins in the first row. In other words, second row of pins 1649 has an empty space between each pin that corresponds to pin 1647 on the first row. Further, the second row of pins 1649 are not positioned at a wire crossing for wires passing over the first row of pins 1647.

FIG. 16I illustrates wire mesh 1650 with two rows of pins, which comprises first set of pins 1651 and second set of pins 1653. The second row of pins are located at two positions relative to the first row of pins. A first position 1654 is substantially in-line with (e.g., in a vertical alignment with) the first row of pins 1651, and a second position 1655 in the second row of pins is in a first “off” position in relation to the first row of pins. In one embodiment, every other pin on the first row has a pin aligned with it on the second row. In one embodiment, there are more pins in the second row than pins in the first row. In one embodiment, as illustrated in FIG. 16I, the ratio of the number of pins in the first to second rows is 5:6.

FIG. 16J illustrates wire mesh 1660 with two rows of pins, which comprises first set of pins 1661 and second set of pins 1663. The second row of pins are located at two positions relative to the first row of pins. A first position 1664 is substantially in-line with (e.g., in a vertical alignment with) the first row of pins, and a second position 1665 is in a second “off” position in relation to the first row of pins. In one embodiment, the second off position 1665 is substantially in the middle of each adjacent pin in the first row of pins 1661. In one embodiment, each pin on the first row has a pin aligned with it on the second row. In one embodiment, there are more pins in the second row than pins in the first row. In one embodiment, the ratio of the number of pins in the first to second rows is 5:10.

FIG. 16K illustrates wire mesh 1670 with two rows of pins, which comprises first set of pins 1671 and second set of pins 1673. The second row of pins are substantially in-line with (e.g., in a vertical alignment with) the first row of pins, but the number of pins in the second row is approximately half as the number of pins in the first row. In other words, the second row of pins has an empty space between each pin that corresponds to a pin on the first row. In one embodiment, every other pin on the first row has a pin aligned with it on the second row. In one embodiment, there are more pins in the first row than pins in the second row. Further, the second row of pins 1673 are not positioned at a wire crossing for wires passing over the first row of pins 1671.

### Deployment of Stent

In one embodiment, the deployment of the disclosed stent follows the principle of the Supera woven stent. For example, it uses a reciprocal mechanism that deploys the stent incrementally. These stents may be stackable, meaning their mesh tightness can be increased during deployment, which is a particularly advantageous feature where the radial force of the stent should be increased locally (such as places where heavily calcified lesions are present). Because the stent deployment is performed by releasing a small increment at a time, the phenomenon of foreshortening is completely eliminated. The repositionability that the delivery system offers makes it easy to correct any malposition of the stent and allows for complete withdrawal of the partially deployed stent back to the delivery catheter. Further, after correcting the position of the delivery system the stent deployment can be repeated.

The disclosed stent may be inserted and deployed in a variety of manners. Similar to the deployment of a Supera based stent, the disclosed stent may be used with a guidewire and delivery catheter. In one example, the size of the delivery catheter may be a 5F, 6F or 7F catheter. Other sizes are possible: for example, for venous stents the delivery system may be 8F-10F. In one embodiment, the disclosed stent may be delivered similar to that described in U.S. Pat. Nos. 8,876,881 and 9,023,095, each incorporated herein by reference.

In one embodiment, the disclosed stent provides deployment capabilities without foreshortening. For example, during deployment, the distal part of the woven nitinol stent anchors firmly itself within the given vascular structure. As the deployment continues more and more length of the stent is getting contact to the vessel wall. In one embodiment, the stent is stackable, that is, the subsequent portions of the stent are deployed continuously behind the already deployed ones. Thus, at the very end of the deployment there is no jump when the very end of the stent exits from the delivery catheter.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention.

Many other variations in the system are within the scope of the invention. For example, the disclosed stent may or may not be deployed by itself. The stent may be substantially straight, conical, and/or tapered, and may have a flanged portion on one end of the stent. In some embodiment, the disclosed stent may be an integral part of one or more stenting systems (whether flanged or straight), and thus may be considered a modular flange system. The disclosed stent may be used in any anatomical structure and is not limited to branching vessels. The disclosed stent may be used in arterial systems and/or venous systems. The disclosed stent may be formed of individual wire strands and/or wire bundles. The wires utilized in the stent may be twisted. The disclosed stent may be formed of a single continuous wire (or wire bundle) or a plurality of wires (or wire bundles). While nitinol may be one shape memory wire used, a variety of other shape memory materials may similarly be utilized. It is emphasized that the foregoing embodiments are only examples of the very many different structural and material configurations that are possible within the scope of the present invention.

Although the invention(s) is/are described herein with reference to specific embodiments, various modifications and changes can be made without departing from the scope of the present invention(s), as presently set forth in the claims below. Accordingly, the specification and figures are to be regarded in an illustrative rather than a restrictive sense, and all such modifications are intended to be included within the scope of the present invention(s). Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements. The terms “coupled” or “operably coupled” are defined as connected, although not necessarily directly, and not necessarily mechanically. The terms “a” and “an” are defined as one or more unless stated otherwise. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements but is not limited to possessing only those one or more elements. Similarly, a method or process that “comprises,” “has,” “includes” or “contains” one or more operations possesses those one or more operations but is not limited to possessing only those one or more operations.

What is claimed is:

1. A self-expanding woven stent, comprising:
a plurality of shape memory wires woven together to form a body of the stent having a first end and a second end, wherein the plurality of shape memory wires comprises a first wire and a second wire, wherein the first wire is arranged substantially parallel to the second wire.

2. The stent of claim 1, wherein the first wire and the second wire are substantially adjacent to each other.

3. The stent of claim 1, wherein the plurality of shape memory wires further comprises a third wire, wherein the first, second, and third wires are substantially parallel to each other.

4. The stent of claim 1, wherein each of the plurality of shape memory wires bend at a plurality of points creating a mesh of substantially perpendicular crossing wires that form a plurality of cells and a plurality of angles.

5. The stent of claim 4, wherein the plurality of angles are approximately 90 degrees.

6. The stent of claim 4, wherein the plurality of angles comprises acute angles.

7. The stent of claim 4, wherein the plurality of angles faces outward.

8. The stent of claim 1, further comprising a plurality of wire connecting pieces that couple free ends of each of the plurality of shape memory wires together.

9. The stent of claim 1, wherein the plurality of shape memory wires comprises a plurality of wire bundles.

10. The stent of claim 1, wherein the body has a tubular shape.

11. The stent of claim 1, wherein the body has a substantially uniform diameter.

12. The stent of claim 1, wherein the body is substantially tapered.

13. The stent of claim 1, wherein the body comprises a flange portion, wherein a diameter of the flange portion is larger than a diameter of the rest of the body.

14. The stent of claim 1, wherein the plurality of shape memory wires is helically twisted.

15. The stent of claim 1, wherein the stent is an arterial stent.

16. The stent of claim 1, wherein the stent is a venous stent.

17. A self-expanding woven stent, comprising:
a body with a first end and a second end,
wherein the body comprises at least one shape memory wire woven together,
wherein the at least one shape memory wire crosses at a plurality of positions to form a plurality of cells and a plurality of angles, wherein the plurality of angles are approximately 90 degrees.

18. The stent of claim 17, wherein the at least one shape memory wire comprises a wire bundle.

19. The stent of claim 17, wherein the at least one shape memory wire comprises a plurality of shape memory wires.

20. The stent of claim 19, wherein each of the plurality of shape memory wires is located substantially parallel to each other.

21. The stent of claim 19, wherein each of the plurality of shape memory wires is located substantially adjacent to each other.

22. A self-expanding woven stent, comprising:
a body with a first end and a second end,
wherein the body comprises at least one shape memory wire woven together, wherein the at least one shape memory wire comprises at least one wire bundle,
wherein the at least one shape memory wire crosses at a plurality of positions to form a plurality of cells and a plurality of angles.

23. The stent of claim 22, wherein the plurality of angles is approximately 90 degrees.

24. The stent of claim 22, wherein the at least one wire bundle comprises a plurality of individual wire strands.

25. The stent of claim 24, wherein each of the plurality of individual wire strands is twisted.

26. The stent of claim 24, wherein each of the plurality of individual wire strands is approximately the same diameter.

27. The stent of claim 24, wherein the plurality of individual wire strands comprises a plurality of different diameters.

28. The stent of claim 22, wherein the at least one wire bundle comprises non-symmetrical wires.

29. The stent of claim 22, wherein the at least one wire bundle comprises a plurality of compressed wires.

30. The stent of claim 22, wherein the at least one wire bundle comprises a plurality of substantially compacted wires.

31. The stent of claim 22, wherein the at least one wire bundle comprises a plurality of substantially compacted wires and a plurality of substantially round wires.

32. The stent of claim 22, wherein the at least one wire bundle comprises one or more microtubings with a platinum core.

33. The stent of claim 22, wherein the at least one wire bundle comprises at least three individual wires.

34. The stent of claim 22, wherein the at least one wire bundle comprises at least seven individual wires.

35. The stent of claim 22, wherein the at least one wire bundle comprises at least 19 individual wires.

36. The stent of claim 22, wherein the at least one wire bundle is twisted.

37. The stent of claim 22, wherein the at least one wire bundle comprises a plurality of individual wire segments, wherein at least one of the pluralities of individual wire segments comprises an inner wire bundle.

38. The stent of claim 22, wherein the at least one wire bundle comprises a plurality of inner wire bundles, wherein each of the inner wire bundles comprises a plurality of individual wires.

39. The stent of claim 22, wherein the at least one wire bundle comprises a first plurality of wires with a first diameter and a second plurality of wires with a second diameter.

40. The stent of claim 22, wherein the at least one wire bundle comprises an inner set of wire bundles and an outer set of wire bundles.

41. The stent of claim 40, wherein the inner set of wire bundles comprises a first set of wires with a first diameter and a second set of wires with a second diameter.

42. The stent of claim 40, wherein the outer set of wire bundles comprises a first set of wires with a first diameter and a second set of wires with a second diameter.

43. The stent of claim 22, wherein the at least one shape memory wire comprises a plurality of shape memory wires, wherein each of the plurality of shape memory wires comprises a wire bundle to form a plurality of wire bundles.

44. The stent of claim 43, wherein each of the plurality of wire bundles is substantially parallel to each other.

45. The stent of claim 43, wherein each of the plurality of wire bundles is substantially adjacent to each other.

46. The stent of claim 43, wherein each of the plurality of wire bundles comprises an inner set of wire bundles and an outer set of wire bundles.

47. The stent of claim 43, wherein the plurality of wire bundles comprises a first plurality of wire bundles with a first diameter and a second plurality of wire bundles with a second diameter.

48. A method of forming a self-expanding stent, the method comprising:
forming a stent by bending at least one shape memory wire at a first plurality of bends to form a mesh, wherein each of the plurality of bends is approximately 90 degrees; and
heat treating the at least one shape memory wire.

49. The method of claim 48, wherein the at least one shape memory wire comprises a wire bundle.

50. The method of claim 48, wherein the at least one shape memory wire comprises a plurality of shape memory wires, further comprising weaving the plurality of shape memory wires together such that the wires cross at an angle of approximately 90 degrees.

51. A method of forming a self-expanding stent, the method comprising:
forming a stent by weaving a plurality of shape memory wires together to form a body with a first end and a second end, wherein the plurality of shape memory wires is substantially parallel to each other.

52. The method of claim 51, wherein the plurality of shape memory wires is substantially adjacent to each other.

53. The method of claim 51, wherein the plurality of shape memory wires comprises at least three wires.

54. The method of claim 51, wherein the plurality of shape memory wires comprises a plurality of wire bundles.

55. The method of claim 51, wherein each of the plurality of shape memory wires crosses each other to form a plurality of cells and a plurality of angles.

56. The method of claim 55, wherein the plurality of angles is approximately 90 degrees.

57. The method of claim 55, wherein the plurality of angles comprises acute angles.

58. The method of claim 55, wherein the plurality of angles faces outward.

59. The method of claim 51, further comprising bending the plurality of shape memory wires around a plurality of protrusions on a template.

60. The method of claim 59, wherein the plurality of protrusions comprises a first plurality of pins and a second plurality of pins.

61. The method of claim 60, wherein the first plurality of pins is located at a first circumferential position on a template and the second plurality of pins is located at a second circumferential position on the template.

62. The method of claim 60, wherein the second plurality of pins is located proximally on the template to the first plurality of pins.

63. The method of claim 60, wherein an amount of the second plurality of pins is approximately half of the amount of the first plurality of pins.

64. The method of claim 60, wherein the second plurality of pins is substantially in-line with the first plurality of pins.

65. The method of claim 60, wherein at least some of the second plurality of pins is located at an off position relative to the first plurality of pins.

66. The method of claim 59, wherein the plurality of protrusions comprises a first plurality of pins located at a first circumferential position, a second plurality of pins located at a second circumferential position, and a third plurality of pins located at a third circumferential position.

67. A method of forming a self-expanding stent, the method comprising:
forming a stent by weaving at least one wire bundle together to form a body with a first end and a second end,
wherein the at least one wire bundle crosses at a plurality of positions to form a plurality of cells and a plurality of angles.

68. The method of claim 67, wherein the at least one wire bundle comprises a plurality of wire bundles.

69. The method of claim 68, wherein the plurality of wire bundles is substantially parallel to each other.

70. The method of claim 67, further comprising twisting the at least one wire bundle during the weaving step.

71. The method of claim 67,
wherein the at least one wire bundle comprises a plurality of wire segments, wherein each of the plurality of wire segments comprises an inner wire bundle,
further comprising twisting the plurality of wire segments together to form the at least one wire bundle.
